(19) United States
(12) Patent Application Publication
(10) Pub. No.: US 2018/0303810 A1
(43) Pub. Date: Oct. 25, 2018

(54) Nimodipine Water-Soluble Derivative, And Preparation Method And Use Thereof

(71) Applicant: Guangzhou Henovcom Bioscience Co., Ltd., Guangzhou (CN)

(72) Inventors: Jiancun Zhang, Guangzhou (CN); Deyao Li, (US); Kun Wang, (US); Yan Liu, (US); Yuanchao Zhang, (US)

(73) Assignee: Guangzhou Henovcom Bioscience Co., Ltd., Guangzhou (CN)

(21) Appl. No.: 15/751,775

(22) PCT Filed: Jul. 29, 2016
(86) PCT No.: PCT/CN2016/092387

§ 371 Date: Jul. 3, 2018

(30) Foreign Application Priority Data
Aug. 11, 2015 (CN) 201510491288.1

(51) Int. Cl. A61K 31/4422; A61K 31/4439; A61K 31/444; A61K 31/375; A61P 9/10

(52) U.S. Cl. CPC A61K 31/4422; A61K 31/4439; A61P 9/10; A61K 31/375; A61K 31/444

(57) ABSTRACT

Provided are a nimodipine water-soluble derivative, and a preparation method and a use thereof, belonging to the field of pharmaceutical chemistry. The nimodipine water-soluble derivative has the structural feature of general formula I and has a relatively high water solubility, and can be converted into nimodipine by an internal enzyme in blood or in the body, so that the nimodipine water-soluble derivative can be used as a nimodipine prodrug and a calcium ion antagonist for treating cardiovascular and cerebrovascular diseases.

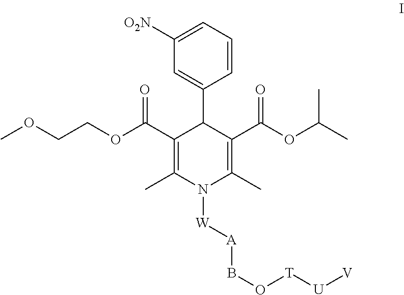

### TECHNICAL FIELD

The present disclosure belongs to the technical field of the pharmaceutical chemistry and relates to a water-soluble Nimodipine derivative and a preparation method and use thereof.

### BACKGROUND

Nimodipine can act on a cerebrovascular smooth muscle with a high degree of selectivity, dilate a cerebral blood vessel, increase a cerebral blood flow, improve a cerebral blood-supply level, and provide a protection on the preclusion of cerebral vascular spasm and a tissue in an ischemic region of cerebral infarction. Clinical research has shown that Nimodipine can regulate calcium ion influx into a nerve cell, affect electrical property of a neuron and the balance of a neurotransmitter. Nimodipine, a cerebrovascular protective agent for people with many cerebrovascular risk factors to pass the critical stage, is commonly used in the prevention and treatment of acute ischemic stroke, and improve the prognosis. Nimodipine is a potent vasodilator and is now an ideal drug mainly for treating a patient with ischemic cerebrovascular disease and a patient with cardio-cerebrovascular disease, without obvious adverse reactions.

However, Nimodipine is a poorly water-soluble drug, and it has characteristics such as low solubility, strong liver first-pass effect and the like, resulting in a low oral bioavailability. Studies have shown that the bioavailabilities of Nimodipine by a healthy subject and a patient with subarachnoid hemorrhage are between 5% and 13% and between 3% and 28%, respectively. The drug has a short biological half-life (about 1.5 to 2 h). The drug needs to be frequently administrated at 3-4 times daily, which is not only inconvenient to use, but also will lead to a “peak-valley” phenomenon in drug concentration in the blood, causing toxic side effects.

Therefore, in practical applications, Nimodipine injection agent can better meet the clinical needs of a patient with cardio-cerebrovascular disease, especially a critically ill patient. However, commercially available Nimodipine injection solution uses a large amount of ethanol as a solvent, which has a great vascular irritation, poor patient compliance, and poor stability for formulation, and prone to drug precipitation, resulting in a severe toxic and adverse reaction.

In order to solve the problem of Nimodipine injection solution in clinical application, Chinese patent No. CN102525917A and Chinese patent No. CN1732936 respectively disclose a Nimodipine micelle injection agents and Nimodipine emulsion injection solutions and preparation methods thereof, avoiding or reducing the use of an organic solvent having a great toxic side effect such as ethanol, ethylene glycol, and the like; Chinese Patent No. CN102274176A discloses a method in which Nimodipine is firstly dissolved in a small volume of organic menstruum and then applied after being mixed with an emulsion; Chinese Patent No. CN101485632 and Chinese Patent No. CN102552156A, in which Nimodipine is made into a liposome, respectively disclose an injection solution of Nimodipine lipid microsphere and a lyophilized solid lipid nanoparticle and preparation methods thereof; and Chinese Patent No. CN1634050 and Chinese Patent No. CN1424035, in which hydroxypropyl-beta-cyclodextrin and cyclodextrin are respectively used in combination with Nimodipine to prepare an inclusion compound, disclose a method of preparing a composition of injection agent of a lyophilized drug of Nimodipine.

Due to the reduction in the amount of organic menstruum, in theory, the above methods can reduce the toxic side effects of Nimodipine injection solution in clinical use to varying degrees, but the means of formulation cannot fundamentally solve the problem of Nimodipine having a poor solubility in water. Due to the intrinsic defects of the formulation, it is very easy to cause the precipitation of the main drug in the cases of long-term placement or large changes in external conditions, which brings a great risk to the clinical use. Therefore, there is still an urgent need to develop a new type of stable Nimodipine prodrug with high solubility in water.

### SUMMARY OF THE INVENTION

In view of the above, it is an object of the present disclosure to provide a water-soluble Nimodipine derivative to overcome the deficiencies of the prior art. Such a derivative is a Nimodipine prodrug with high solubility in water and can be rapidly transformed to Nimodipine through the intrinsic enzymes in blood or in vivo.

In order to achieve the above object, the present disclosure adopts the following technical solution:

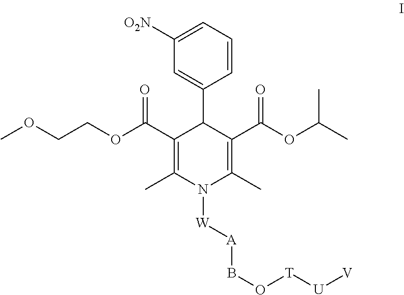

wherein:

W is selected from C═O, C═S, or SO₂; or W is absent;

A is selected from O or S; or A is absent;

B is C(R₄)(R₅), or absent;

each of R₄ and R₅ is independently selected from hydrogen, deuterium, C₁-C₃ alkyl, C₁-C₃ alkyl substituted by R₁₅, aryl, or aryl substituted by R₁₅, and R₄ and R₅ can form a 4 to 6-membered ring with each other;

R₁₅ is selected from O, carboxyl, or amino;

T is selected from C═O, SO₂, SO₃R₆, PO₃R₇R₈, or PO₂R₁₇(NHR₁₈); or T is absent;

each of R₆, R₇, and R₈ is independently selected from H, a metal ion, or an ammonium ion;

R₁₇ is selected from aryl, substituted aryl, naphthyl, or substituted naphthyl;

NHR₁₈ is an amino acid group;

U is selected from C₁-C₈ alkyl, carboxyl-containing C₁-C₈ alkyl, C₃-C₈ cycloalkyl, aryl, alkenyl, alkynyl, nitrogen-containing heterocycloalkyl, guanidyl-containing C₁-C₈ alkyl, amide-containing C₁-C₈ alkyl, 2-4 peptide alkyl, C₁-C₈ alkyl substituted by R₁₆, C₃-C₈ cycloalkyl substituted by R₁₅, aryl substituted by R₁₅, alkenyl substituted by R₁₅, or alkynyl substituted by R₁₅; or U is absent;

R₁₆ is selected from amino, carboxyl, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;

V is selected from NR₉R₁₀, COOR₁, PO₃R₁₂R₁₃ or SO₃R₁₄; or V is absent;

each of R₉ and R₁₀ is independently selected from hydrogen, C₁-C₈ alkyl, or C₁-C₈ alkyl substituted by R₁₅, and R₉ and R₁₀ can form a 4 to 8-membered ring with each other;

each of R₁₁, R₁₂, R₁₃, and R₁₄ is independently selected from H, a metal cation, or an ammonium ion; and

the metal cation is selected from sodium ion, potassium ion, lithium ion, calcium ion, or magnesium ion.

In one embodiment, the water-soluble Nimodipine derivative is selected from a structure represented by the following formula II:

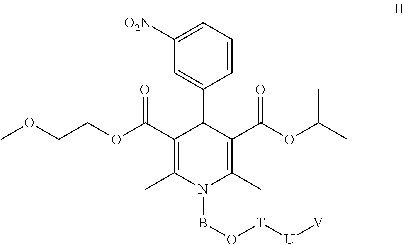

B is C(R₄)(R₅); and

each of R₄ and R₅ is independently selected from hydrogen, deuterium, or C₁-C₃ alkyl.

In one embodiment, the water-soluble Nimodipine derivative is selected from a structure represented by the following formula III:

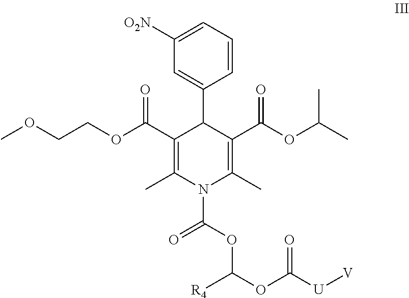

R₄ is selected from hydrogen, deuterium, or C₁-C₃ alkyl.

In one embodiment, U is selected from C₁-C₈ alkyl, alkenyl, or C₁-C₈ alkyl substituted by R₁₆;

R₁₆ is selected from amino, or carboxyl;

V is selected from NR₉R₁₀, or COOR₁₁; or V is absent; and

each of R₉ and R₁₀ is independently selected from hydrogen, or C₁-C₈ alkyl.

In one embodiment, U is selected from C₁-C₈ alkyl, alkenyl, nitrogen-containing heterocycloalkyl, guanidyl-containing C₁-C₈ alkyl, amide-containing C₁-C₈ alkyl, 2-4 peptide alkyl, or C₁-C₈ alkyl substituted by R₁₆; or U is absent;

R₁₆ is selected from amino, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;

V is selected from NR₉R₁₀, COOR₁₁ or PO₃R₁₂R₁₃; or V is absent; and

each of R₉ and R₁₀ is independently selected from hydrogen, or C₁-C₈ alkyl.

In one embodiment, U together with V can form one of the following groups:

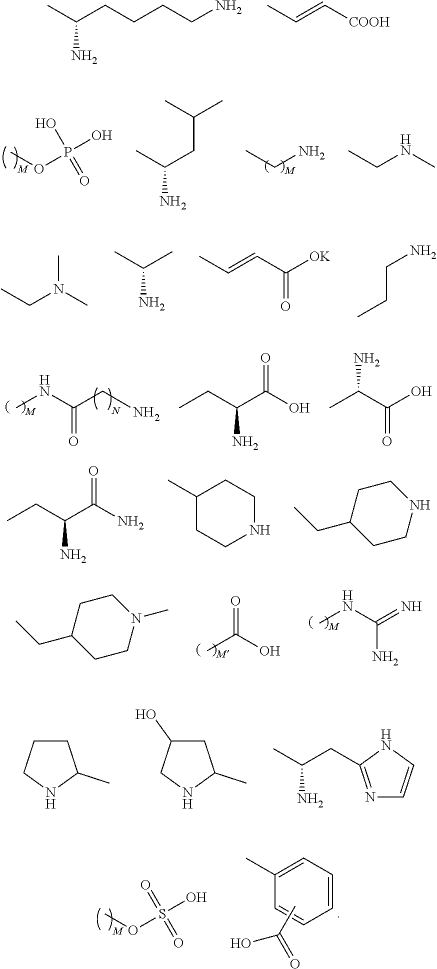

In one embodiment, the water-soluble Nimodipine derivative is selected from a structure represented by the following formula IV:

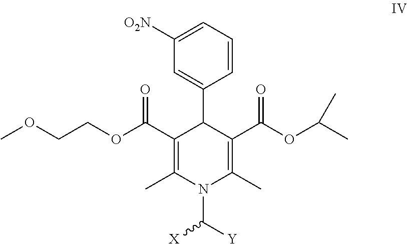

wherein:

when X is H, Y is selected from OH

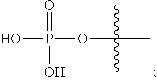 [SMILES: *OP(=O)(O)O]

when X is ═O, Y is selected from

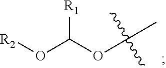

R₁ is selected from hydrogen, C₁-C₆ alkyl, or substituted C₁-C₆ alkyl;

R₂ is selected from one of the following groups:

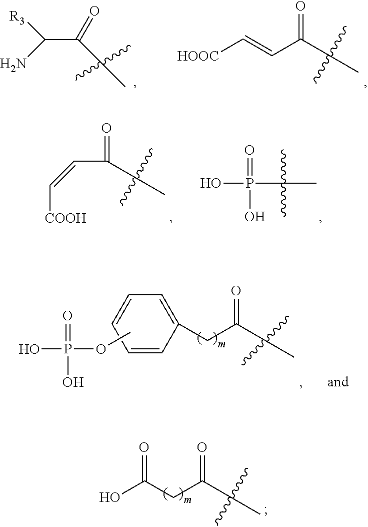

R₃ is selected from hydrogen, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;

m is selected from 0, 1, 2, or 3; and

n is selected from 0, 1, or 2.

In one embodiment, the natural amino acid is selected from lysine, arginine, or histidine.

In one embodiment, the pharmaceutically acceptable salt is selected from sodium salt, potassium salt, calcium salt, magnesium salt, lithium salt, lysine salt, arginine salt, aspartic acid, glutamic acid, tromethamine salt, ethanolamine salt, hydrochloride, sulfate, phosphate, citrate, acetate, maleate, lactate, methanesulfonate, oxalate, fumarate, hydrobromide, p-toluenesulfonate, benzenesulfonate, or nitrate.

In one embodiment, R₁ is hydrogen; and R₃ is a side chain group of a natural amino acid.

In one embodiment, R₁ is selected from hydrogen, or Me; and U together with V can form one of the following groups:

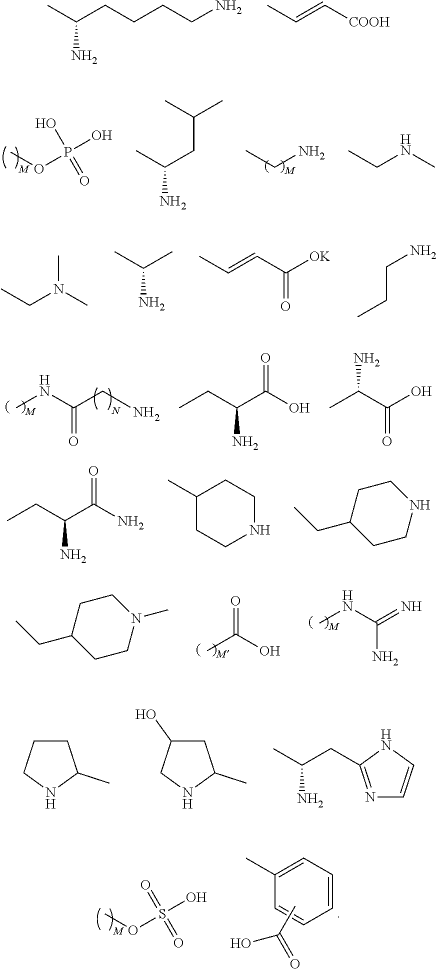

In one embodiment, the water-soluble Nimodipine derivative is selected from one of the following compounds:

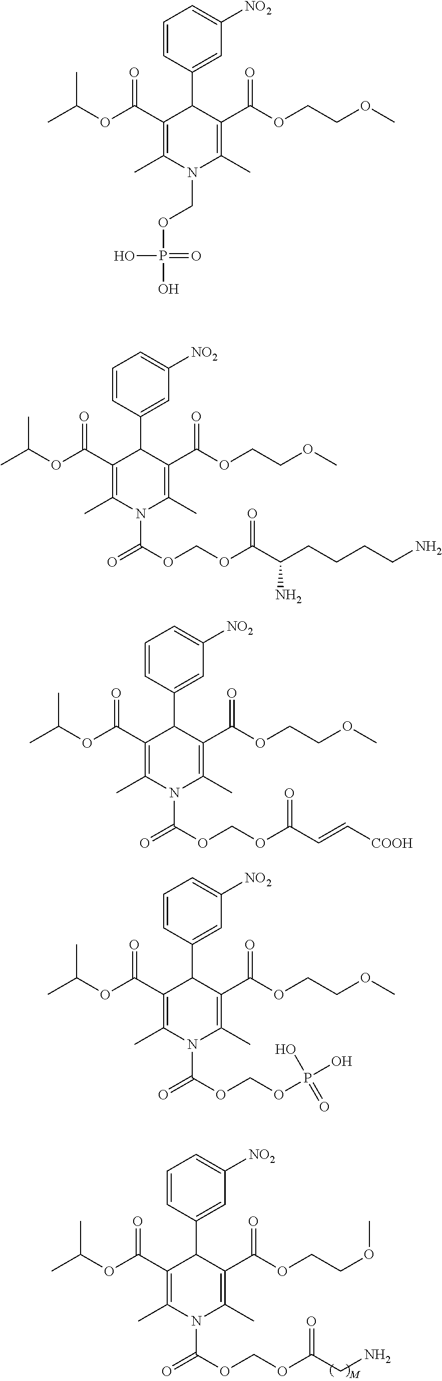

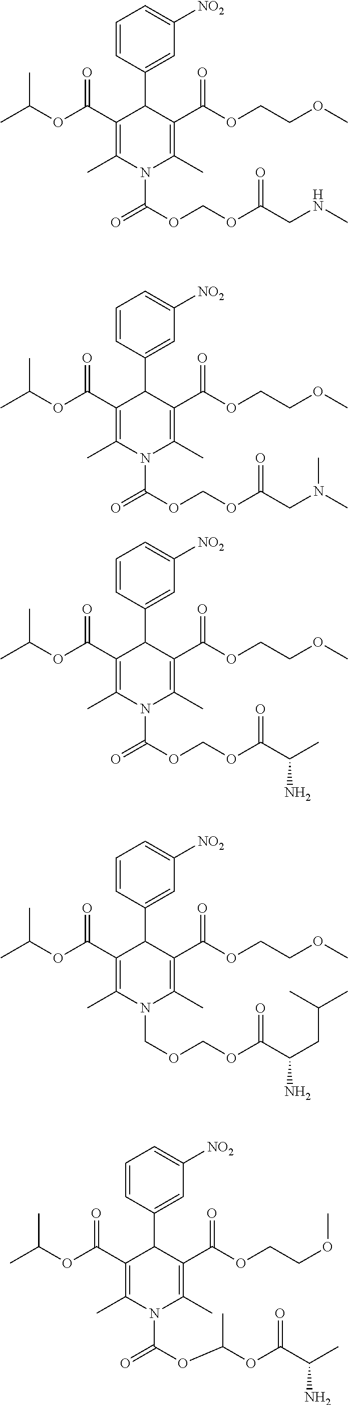

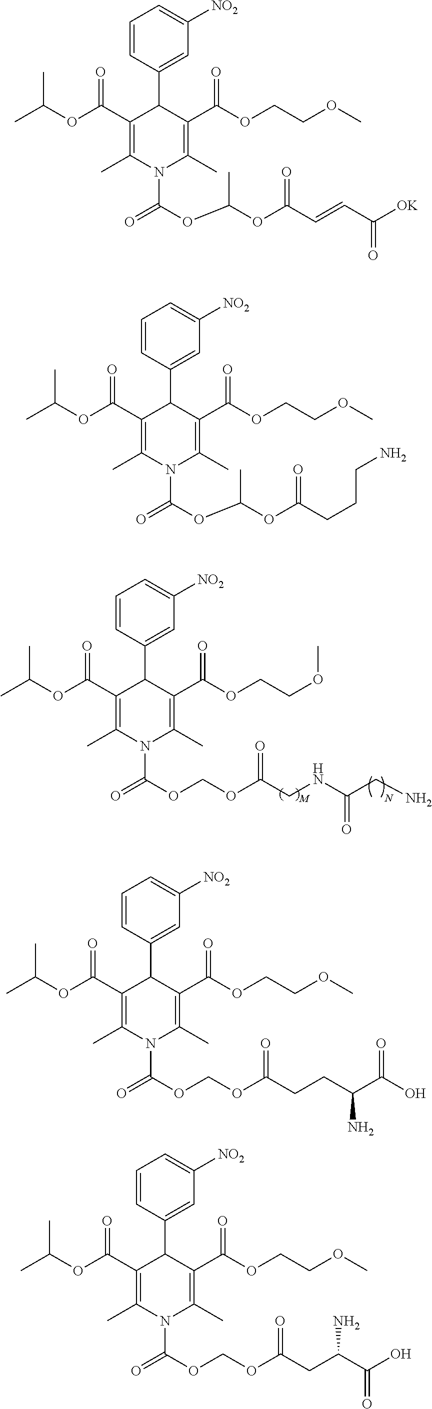

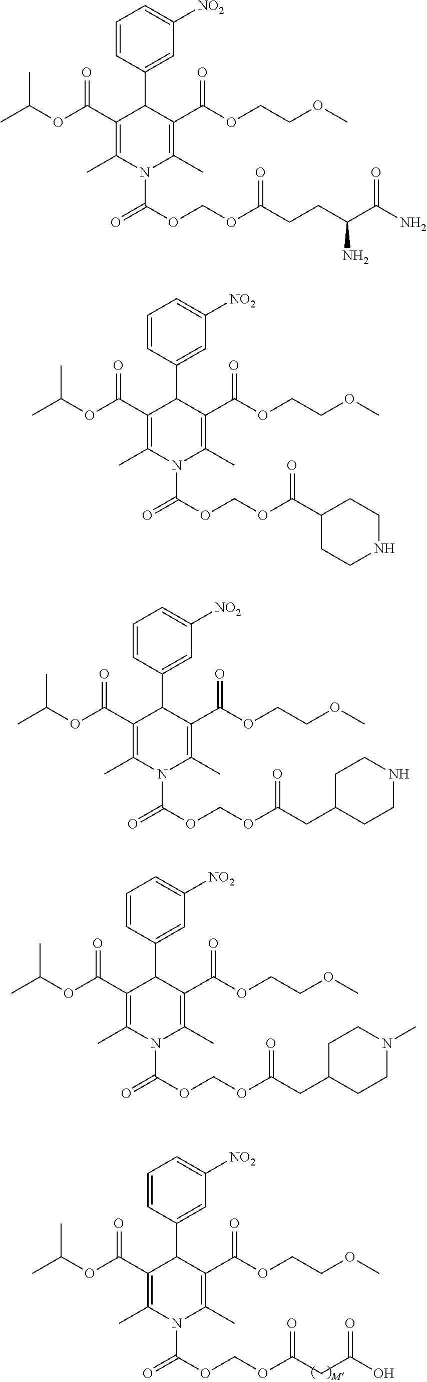

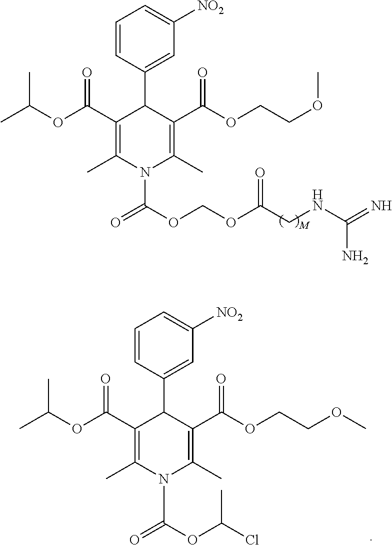

The present disclosure further discloses a method of preparing the above-mentioned water-soluble Nimodipine derivative, comprising the following steps: reacting Nimodipine with halogenate chloroformate to form an amide; then reacting amide with a corresponding carboxylic acid, amino acid or phosphoric acid derivative to form an ester; and deprotecting, so as to yield the water-soluble Nimodipine derivative; and the reaction route is shown as below:

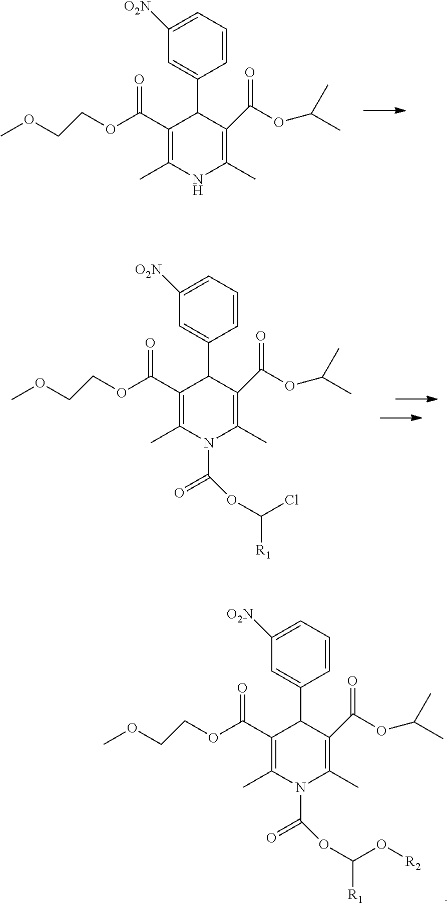

Alternatively, the method comprises the following steps: reacting Nimodipine with di-tert-butyl chloromethyl phosphate to form a methylene phosphate; then deprotecting, so as to yield the water-soluble Nimodipine derivative. The reaction route is shown as below:

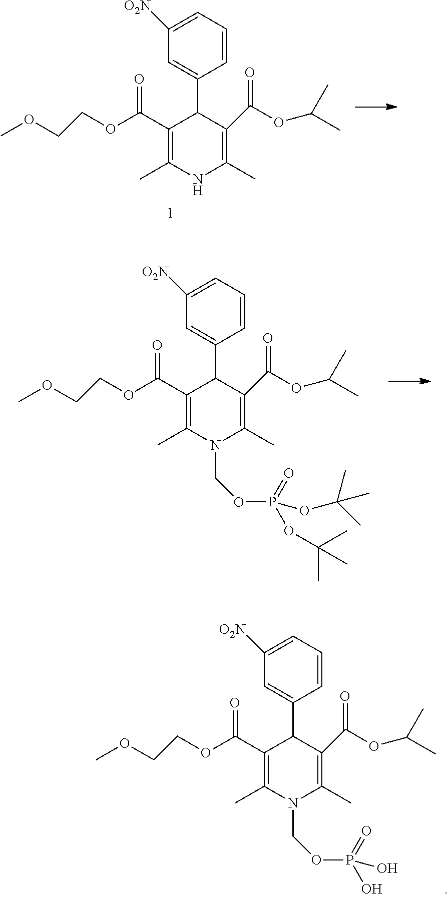

The present disclosure further discloses a method of preparing a pharmaceutically acceptable salt of the above-mentioned water-soluble Nimodipine derivative, which is characterized in that the above-mentioned water-soluble Nimodipine derivative is reacted with an acid or a base to form a salt, so as to yield the pharmaceutically acceptable salt.

The present disclosure further discloses the use of the above-mentioned water-soluble Nimodipine derivative or a pharmaceutically acceptable salt thereof in the preparation of cardio-cerebrovascular drugs.

The present disclosure further discloses a pharmaceutical composition for treating a cardio-cerebrovascular disease, comprising the above-mentioned water-soluble Nimodipine derivative or a pharmaceutically acceptable salt thereof as an active ingredient, and a pharmaceutically acceptable carrier.

The above-mentioned pharmaceutically acceptable carrier refers to a pharmaceutical carrier commonly used in the pharmaceutical field, for example, an excipient; polyalcohols, such as mannitol, sorbitol, inositol, and xylitol; sugars such as glucose, dextran, lactose, maltose, raffinose, fructose, etc.; an antioxidant, such as sodium bisulfite, sodium metabisulfite, vitamin C, vitamin E, etc.; a complexing agent, such as EDTA-2Na (ethylenediaminetetraacetic acid disodium), etc.; an isoosmotic adjusting agent, such as: sodium chloride, potassium chloride, etc.; a solubilizer, such as Tween 80, etc.; a menstruum for injection, such as water, propylene glycol, glycerin, etc.; a topical analgesic, such as benzyl alcohol, etc.; an antibacterial agent, such as nipagins, etc.; a pH regulator, such as hydrochloric acid, sodium hydroxide, sodium carbonate, sodium bicarbonate, citric acid, sodium citrate and the like.

Compared with the prior art, the present disclosure has the following advantages:

The water-soluble Nimodipine derivative or the pharmaceutically acceptable salt thereof having the structural feature of formula I of the present disclosure utilizes the chemically reaction with imine group in Nimodipine to transform Nimodipine into a series of derivatives containing an acid or an amino group, and thus preparing Nimodipine prodrug with high solubility in water. An acid or an amino group is further used to form a salt, greatly improving the solubility of such water-soluble Nimodipine derivatives, all of which exhibit water-soluble characteristics and each with a solubility of more than 50 mg/mL and over 10,000-fold than that of Nimodipine in water.

Moreover, when such prodrug enters the body, it can be quickly metabolized into Nimodipine through the intrinsic enzymes (such as phosphatases and esterases) in the blood or in vivo, resulting in corresponding pharmacological activities. The half-life t_{1/2} of rapidly metabolization of the prodrug into Nimodipine is between 0.5 minutes and 2.5 hours. Therefore, the water-soluble Nimodipine derivative of the present disclosure, when is administrated as a drug for treating cardio-cerebrovascular diseases, can both ensure efficacy and reduce side effects in clinical use.

### DETAILED DESCRIPTION OF THE EMBODIMENTS

In the compounds described herein, the line drawn from the substituent into the ring system indicates that the indicated bond can be linked to any ring atom that can be substituted.

The term “alkyl” as used herein is intended to include a branched and straight chain saturated aliphatic hydrocarbon group having a particular number of carbon atoms. For example, the definition of “C₁-C₆” in “C₁-C₆ alkyl” includes a group having 1, 2, 3, 4, 5, or 6 carbon atoms arranged in a straight chain or a branched chain. For example, “C₁-C₆ alkyl” specifically includes methyl, ethyl, n-propyl, isopropyl, n-butyl, tert-butyl, isobutyl, pentyl, and hexyl.

The term “cycloalkyl” refers to a saturated monocyclic aliphatic hydrocarbon group having a specific number of carbon atoms. For example, “cycloalkyl” includes cyclopropyl, methyl-cyclopropyl, 2, 2-dimethyl-cyclobutyl, 2-ethyl-cyclopentyl, cyclohexyl, and the like.

The term “aryl” refers to a carbocyclic aromatic system containing one or two rings, wherein the rings may be linked together in a fused fashion, and include an aromatic group such as phenyl, naphthyl, indenyl, tetrahydronaphthyl and indanyl. The more preferred aryl is phenyl.

The term “alkenyl” refers to a hydrocarbon group having an unsaturated alkenyl group, such as —CH═CH—.

The term “alkynyl” refers to a hydrocarbon group having an unsaturated alkynyl group, such as —C═C—.

The term “heterocyclyl groups” includes saturated heteroatom-containing cycloalkyl and heteroaryl, wherein the heteroatom may be selected from nitrogen, sulfur, and oxygen, and any oxidation state forms of the nitrogen, sulfur, and phosphorus.

Examples of saturated heterocycloalkyl groups include a 3 to 8-membered saturated heteromonocyclic group containing 1 to 4 nitrogen atoms, such as pyrrolidinyl, imidazolidinyl, piperidinyl, pyrrolinyl, piperazinyl; a 3 to 8-membered saturated heteromonocyclic group containing 1 to 2 oxygen atoms and 1 to 3 nitrogen atoms, such as morpholinyl; a 3 to 8-membered saturated heteromonocyclic group containing 1 to 2 sulfur atoms and 1 to 3 nitrogen atoms, such as thiazolidinyl. “Nitrogen-containing heterocycloalkyl” refers to a 3 to 8-membered saturated heteromonocyclic group containing 1 to 4 nitrogen atoms.

The term “amide-containing alkyl” refers to a branched or straight chain hydrocarbon group including an amide linkage and having a specific number of carbon atoms, for example isoglutamine, dipeptide alkyl, tripeptide alkyl, and the like.

The term “alkyl containing 2 to 4 peptides” refers to a small molecule peptide consisted of 2 to 4 amino acids, preferably a small molecule peptide consisted of 2 amino acids.

The term “guanidyl-containing alkyl” refers to alkyl substituted by guanidyl.

Examples of “heteroaryl” include a 5 to 8-membered unsaturated heteromonocyclic group containing 1 to 4 nitrogen atoms, such as pyrrolyl, imidazolyl, pyrazolyl, 2-pyridyl, 3-pyrazolyl, 4-pyridyl, pyrimidyl, pyrazinyl, and pyridazinyl; triazolyl, such as 4H-1,2,4-triazolyl, 1H-1,2,3-triazolyl, 2H-1,2,3-triazolyl; a 5 to 8-membered unsaturated heteromonocyclic group containing one oxygen atom, such as pyranyl, 2-furyl, 3-furyl, and the like; a 5 to 8-membered unsaturated heteromonocyclic group containing one sulfur atom, such as 2-thienyl, 3-thienyl, and the like; a 5 to 8-membered unsaturated heteromonocyclic group containing 1 to 2 oxygen atoms and 1 to 3 nitrogen atoms, such as oxazolyl, isoxazolyl, and oxadiazolyl; a 5 to 8-membered unsaturated heteromonocyclic group containing 1 to 2 sulfur atoms and 1 to 3 nitrogen atoms, such as thiazolyl, thiadiazolyl.

Particular examples of nitrogen-free heteroaryl include pyranyl, 2-furyl, 3-furyl, 2-thienyl, 3-thienyl, benzofuranyl, benzothienyl, and the like.

Cycloalkyl group may contain 3 to 20 ring-forming atoms and may be either monocyclic or polycyclic if an appropriate number of atoms that are membered a ring is present. Examples of cycloalkyl group are cyclopropyl, cyclopentyl, cyclohexyl and adamantyl groups.

The term “substituted” means that hydrogen on the carbon chain is substituted with halogen (ie, fluorine, chlorine, bromine or iodine atom) or an amino group. For example, “substituted C₁-C₃ alkyl” means chloromethyl, bromoethyl, 3-chloropropyl, 4-chlorobutyl, and the like.

The term “a side chain group of a natural amino acid” means an alkyl group, aryl group, heterocyclic moiety linked to the alpha carbon atom of an amino acid. For example, a side chain group of lysine, arginine or histidine refers to one of the following groups:

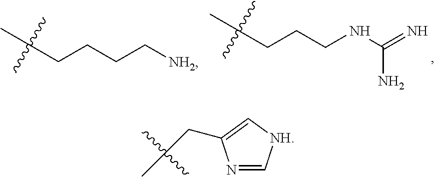 [SMILES: *CCCCN ; *CCCNC(=N)N ; "," ; *Cc1c[nH]cn1]

The term “absent” means that there is no substituent listed in the general formula of the structural formula, and the two moieties adjacent to the substituent are directly linked, as Formula II is the case in which W and A are selected as “absent”.

In addition to the standard methods that are known from the literature or exemplified in laboratory procedures, the compound of the present disclosure can be prepared by the reactions shown in the following schemes. Accordingly, the following illustrative schemes are shown for the purpose of illustration and will not be limited to the listed compounds or any specific substituents, and the methods are intended to be merely illustrative and not limit to the scope of the present disclosure.

Wherein THF refers to tetrahydrofuran, TLC refers to thin layer chromatography, PE refers to petroleum ether, EA refers to ethyl acetate, DMF refers to dimethylformamide, TBAI refers to tetrabutylammonium iodide, DCM refers to dichloromethane, Boc refers to tert-butyloxycarbonyl, and eq refers to equivalent.

### Example 1 Preparation of Compound 3

The reaction was preformed according to the following route:

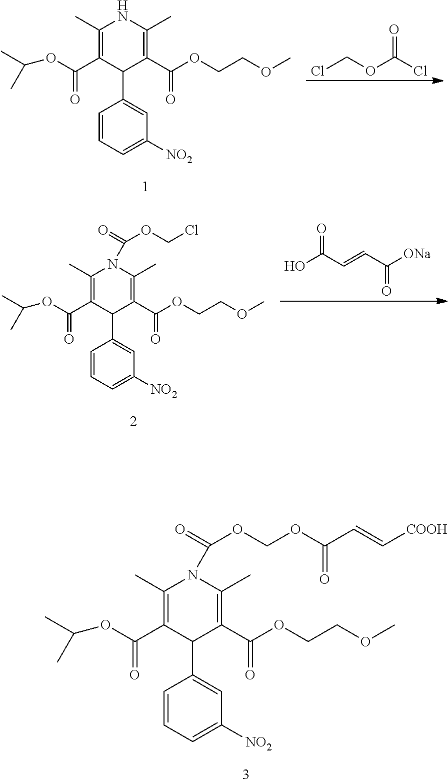

### (1) Preparation of Compound 2

To a solution of dry THF was added NaH (10 g, 240 mmol), followed by 300 mL of Nimodipine (compound 1) in THF (50 g, 120 mmol) in dropwise in an ice bath under nitrogen atmosphere. After half an hour, chloromethyl chloroformate (15 mL, 150 mmol) was added dropwise. The reaction was warmed to room temperature. After completion of the reaction (monitored by TLC), a saturated solution of ammonium chloride was added, and then the reaction mixture was extracted with EtOAc (ethyl acetate), washed once with saturated brine, dried over anhydrous sodium sulfate, concentrated, and purified by column chromatography (PE/EA=3:1) to afford compound 2 as yellow oil (57.9 g, 95%).

The characterization data for the product: ¹H-NMR (400 MHz, CDCl₃) (8.06 (s, 1H, H-2), 8.03 (d, 1H, J=8.0 Hz, H-4), 7.60 (d, 1H, J=8.0 Hz, H-6), 7.37 (t, 1H, J=8.0 Hz, H-5), 5.79 (s, 2H, CH₂), 5.28 (s, 1H, H-4), 5.10 (m, 1H, CH(CH₃)₂), 4.37-4.11 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.32-1.24 (m, 6H, CH(CH₃)₂).

### (2) Preparation of Compound 3

To a flask was added compound 2 (2.15 g, 4.2 mmol), followed by monosodium fumarate (1.2 eq) and 20 mL of DMF. The mixture was heated at 120° C. for 3 hours, and then rotary evaporated for removal of DMF. The residue was purified by column chromatography (PE/EA=2:1) to afford compound 3 as yellow oil (1.34 g, 54%).

The characterization data for the product: ¹H-NMR (400 MHz, CDCl₃) δ 8.02 (m, 2H, H-2, H-4), 7.60 (d, 1H, J=8.0 Hz, H-6), 7.37 (t, 1H, J=8.0 Hz, H-5), 6.89 (m, 2H), 5.92 (s, 2H, CH₂), 5.30 (s, 1H, H-4), 5.10 (m, 1H, CH(CH₃)₂), 4.37-4.11 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.32-1.24 (m, 6H, CH(CH₃)₂).

### Example 2 Preparation of Compound 6

The reaction was preformed according to the following route:

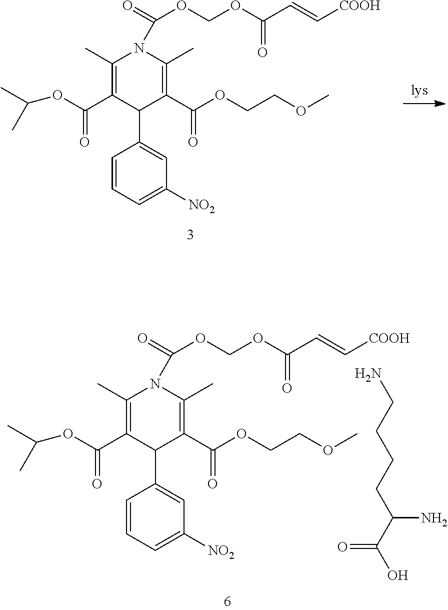

Compound 3 (590 mg, 1 mmol) was dissolved in 5 mL of ethanol, and 0.5 mL of lysine aqueous solution (1.5 eq) was added dropwise at 40° C. After 0.5 h, 10 mL of ethanol was added. The reaction was cooled to 0° C. overnight. The reaction solution was filtered to afford compound 6 as a white solid (210 mg, 35%).

Compound 6 has a solubility of 120 mg/mL in water at room temperature. A portion of compound 6 was throughly mixed with rat anticoagulated plasma and incubated at 37° C. The drug was extracted with acetonitrile at different time points for HPLC analysis. The half-life for converting compound 6 into Nimodipine in blood was determined to be approximately 1.5 hour.

### Example 3 Preparation of Compound 7

The reaction was preformed according to the following route:

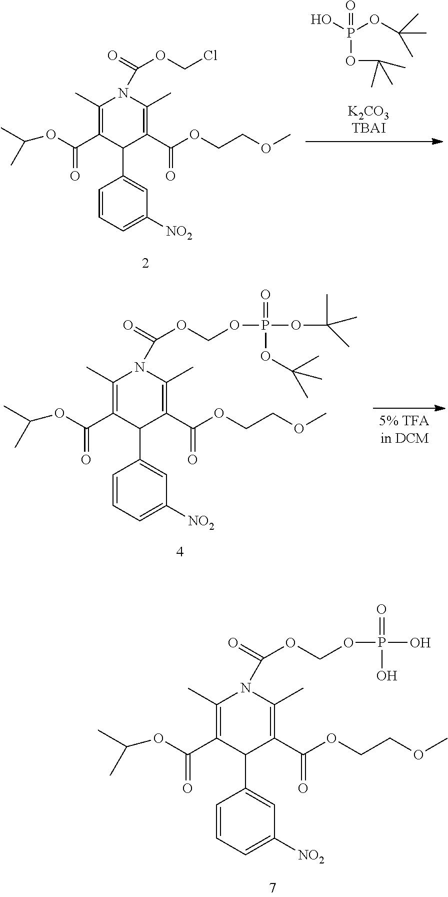

### (1) Preparation of Compound 4

To a 100 mL reaction flask were added compound 2 (2.15 g, 4.2 mmol), potassium carbonate (2 eq), and TBAI 0.2 eq, followed by 25 mL of dry 1,4-dioxane. Di-tert-butyl phosphate (1.6 eq) was added under nitrogen atmosphere and the mixture was reacted at 80° C. overnight. After completion of the reaction (monitored by TLC), the reaction mixture was extracted with ethyl acetate, washed once with saturated brine, dried over anhydrous sodium sulfate, filtered, concentrated, and purified by column chromatography (PE:EA=3:1) to afford compound 4 as yellow oil (2.52 g, 87%).

The characterization data for the product: ¹H-NMR (400 MHz, CDCl₃) δ 8.06 (s, 1H, H-2), 8.03 (d, 1H, J=8.0 Hz, H-4), 7.56 (d, 1H, J=8.0 Hz, H-6), 7.42 (t, 1H, J=8.0 Hz, H-5), 5.68, 5.65 (2s, 1H, CH₂), 5.29 (s, 1H, H-4), 5.10 (m, 1H, CH(CH₃)₂), 4.37-4.11 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.47 (s, 18H, tBu), 1.32-1.24 (m, 6H, CH(CH₃)₂).

### (2) Preparation of Compound 7

Compound 4 (2 g, 2.9 mmol) was dissolved in 15 mL of 5% TFA in DCM solution in an ice bath. After the reaction was completed, the reaction mixture was concentrated in vacuo, toluene was added and then concentrated (repeat twice) to afford compound 7 as yellow oil (1.88 g, 95%).

The characterization data for the product: ¹H-NMR (400 MHz, CDCl₃) δ 9.04 (brs, 2H, OH), 8.04 (s, 1H, H-2), 7.98 (d, 1H, J=8.0 Hz, H-4), 7.53 (d, 1H, J=8.0 Hz, H-6), 7.40 (t, 1H, J=8.0 Hz, H-5), 5.70, 5.66 (2s, 1H, CH₂), 5.29 (s, 1H, H-4), 5.10 (m, 1H, CH(CH₃)₂), 4.39-4.28 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.32-1.24 (m, 6H, CH(CH₃)₂).

### Example 4 Preparation of Compound 8

The reaction was preformed according to the following route:

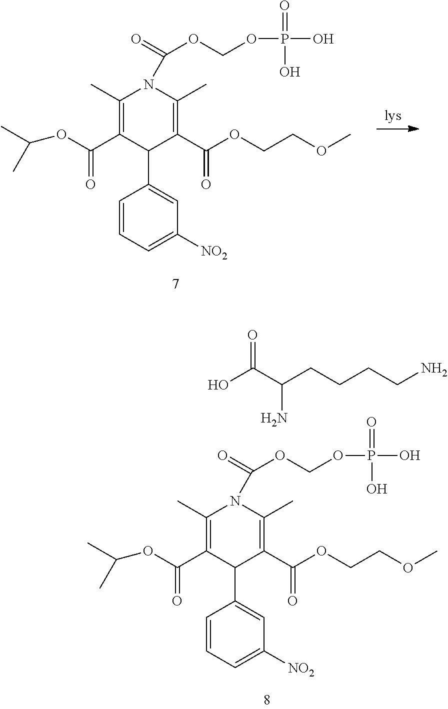

Compound 7 (1 g, 1.4 mmol) was dissolved in 7 mL of ethanol, and 0.7 mL of lysine aqueous solution (1.5 eq) was added dropwise at 40° C. After 0.5 h, 14 mL of ethanol was added, and the reaction was cooled to 0° C. overnight. The reaction solution was filtered to afford compound 8 as a white solid (633 mg, 63%).

Compound 8 has a solubility of 105 mg/mL in water at room temperature. A portion of compound 8 was throughly mixed with rat anticoagulated plasma and incubated at 37° C. The drug was extracted with acetonitrile at different time points for HPLC analysis. The half-life for converting compound 8 into Nimodipine in blood was determined to be approximately 1.5 hour.

### Example 5 Preparation of Compound 9

The reaction was preformed according to the following route:

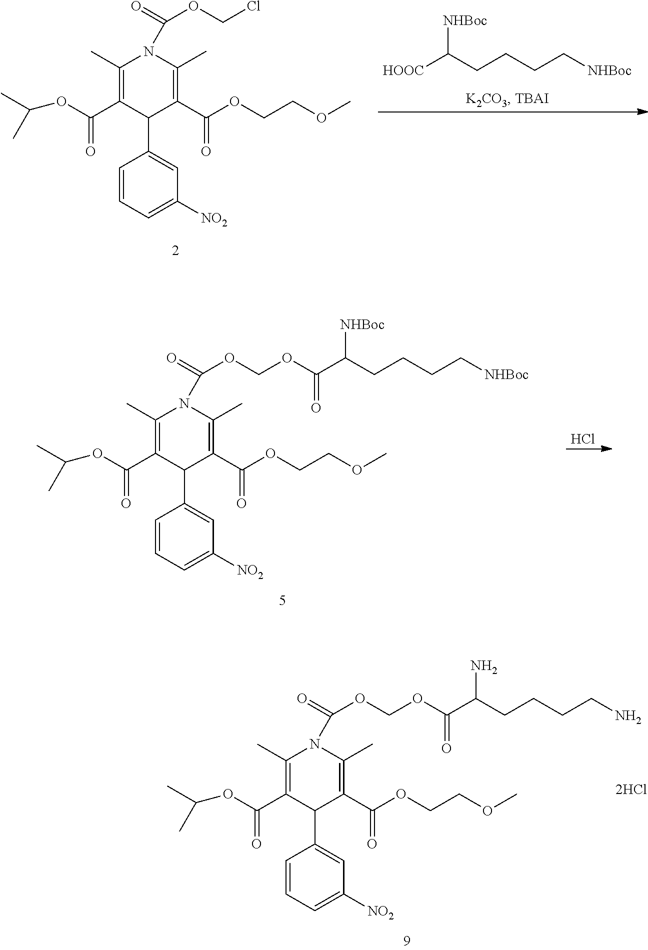 [SMILES: COCCOC(=O)C1=C(C)N(C(=O)OCCl)C(C)=C(C(=O)OC(C)C)C1c1cccc([N+](=O)[O-])c1 ; CC(C)(C)OC(=O)NCCCCC(NC(=O)OC(C)(C)C)C(=O)O ; COCCOC(=O)C1=C(C)N(C(=O)OCOC(=O)C(CCCCNC(=O)OC(C)(C)C)NC(=O)OC(C)(C)C)C(C)=C(C(=O)OC(C)C)C1c1cccc([N+](=O)[O-])c1 ; Cl ; COCCOC(=O)C1=C(C)N(C(=O)OCOC(=O)C(N)CCCCN)C(C)=C(C(=O)OC(C)C)C1c1cccc([N+](=O)[O-])c1]

### (1) Preparation of Compound 5

To a reaction flask were added potassium carbonate (276 mg, 2 mmol), TBAI (73 mg, 0.2 mol), and compound 2 (449 mg, 1.3 eq), followed by 6 mL of 1,4-dioxane. The reaction solution was stirred for 0.5 h at room temperature. Boc-protected lysine (510 mg, 1 mmol) was added and the reaction was performed at 90° C. for 3 h. After completion of the reaction (monitored by TLC), the reaction solution was cooled to room temperature, extracted with EA, washed once with saturated NaCl, dried, filtered, concentrated, and the residue was purified by column chromatography (PE:EA=3:1) to afford compound 5 as yellow oil (680 mg, 83%).

The characterization data for the product: ¹H-NMR (400 MHz, CDCl₃) δ 8.03 (m, 2H, ArH), 7.58 (d, 1H, J=8.0 Hz, ArH), 7.40 (t, 1H, ArH), 5.87 (m, 2H, OCH₂O), 5.29 (s, 1H, H-4), 5.09 (m, 2H, OCH(CH₃)₂, H-1_{1ys}), 4.60 (s, 1H, NH), 4.39-4.26 (m, 3H, OCH₂, NH), 3.63 (m, 2H, OCH₂), 3.36 (s, 1H, OMe), 3.08 (2H, H-4_{1ys}), 2.51 (2s, 6H, Me), 1.64-1.23 (m, 30H, 3CH₂, 2Boc, 2Me).

### (2) Preparation of Compound 9

Compound 5 (820 mg, 1.0 mmol) was dissolved in 4 mL of EA, and 1 mL of hydrochloric acid was added. The reaction was carried out at room temperature for two hours. After completion of the reaction (monitored by TLC), the solvent was evaporated and ethyl ether was added, then a white precipitate was formed, filtered to afford compound 9 as a white solid (630 mg, 82%).

The characterization data for the product: ¹H NMR (400 MHz, DMSO) δ 8.75 (s, 2H, NH₂), 8.12 (d, 1H, ArH), 8.02 (s, 2H, NH₂), 7.90 (s, 1H, ArH), 7.62 (t, 1H, ArH), 7.53 (d, 1H, ArH), 5.88 (s, 2H, OCH₂O), 5.23 (s, 1H, H-4), 5.00 (s, 1H, OCH(CH₃)₂, 4.30 (m, 2H, OCH₂), 4.08 (s, 1H, H-1_{1ys3}), 3.57 (m, 2H, OCH₂), 3.25 (s, 1H, OMe), 2.73 (2H, H-4_{1ys}), 2.50 (2s, 6H, Me), 1.84-1.64 (m, 6H, 3CH₂), 1.48 (m, 6H, 2Me).

Compound 9 has a solubility of 230 mg/mL in water at room temperature. A portion of compound 9 was throughly mixed with rat anticoagulated plasma and incubated at 37° C. The drug was extracted with acetonitrile at different time points for HPLC analysis. The half-life for converting compound 9 into Nimodipine in blood was determined to be approximately 2.5 hours.

### Example 6 Preparation of Compound 11

The reaction was preformed according to the following route:

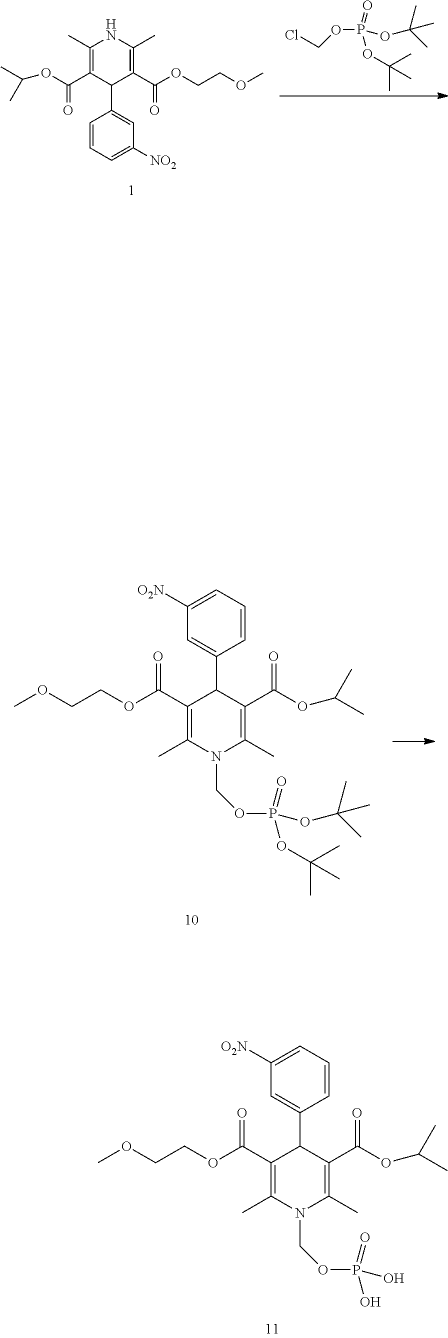

### (1) Preparation of Compound 10

To a solution of dry THF was added NaH (1 g, 25 mmol), followed by 30 mL of Nimodipine (compound 1) in THF (5 g, 12 mmol) and TBAI (0.44 g, 1.2 mmol) in dropwise in an ice bath under nitrogen atmosphere. After half an hour, di-tert-butyl chloromethyl phosphate (3.7 g, 14 mmol) was added dropwise. The reaction was warmed to room temperature for 5 h. After completion of the reaction (monitored by TLC), a saturated solution of ammonium chloride was added, and then the reaction mixture was extracted with EtOAc, washed once with saturated brine, dried over anhydrous sodium sulfate, concentrated, and the residue was purified by column chromatography (PE/EA=3:1) to afford compound 10 as yellow oil (4.2 g, 55%).

The characterization data for the product: ¹H-NMR (400 MHz, CDCl₃) δ 8.07 (s, 1H, H-2), 8.03 (d, 1H, J=8.0 Hz, H-4), 7.54 (d, 1H, J=8.0 Hz, H-6), 7.41 (t, 1H, J=8.0 Hz, H-5), 5.29 (s, 1H, H-4), 5.39 (s, 2H) 5.09 (m, 1H, CH(CH₃)₂), 4.37-4.11 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.47 (s, 18H, tBu), 1.32-1.24 (m, 6H, CH(CH₃)₂).

### (2) Preparation of Compound 11

Compound 10 (2 g, 3.1 mmol) was dissolved in 15 mL of 5% TFA in DCM solution in an ice bath. After the reaction was completed, the reaction mixture was concentrated in vacuo, toluene was added and then concentrated (repeat twice) to afford compound 11 as yellow oil (1.58 g, 96%).

The characterization data for the product: ¹H-NMR (400 MHz, CDCl₃) δ 9.04 (brs, 2H, OH), 8.03 (s, 1H, H-2), 7.97 (d, 1H, J=8.0 Hz, H-4), 7.54 (d, 1H, J=8.0 Hz, H-6), 7.41 (t, 1H, J=8.0 Hz, H-5), 5.38 (s, 2H, CH₂), 5.29 (s, 1H, H-4), 5.10 (m, 1H, CH(CH₃)₂), 4.39-4.28 (m, 2H, OCH₂CH₂O), 3.63 (s, 2H, OCH₂CH₂O), 3.36 (s, 3H, OMe), 2.56 (s, 3H, Me), 2.53 (s, 3H, Me), 1.32-1.24 (m, 6H, CH(CH₃)₂).

### Example 7 Preparation of Compound 12

The reaction was preformed according to the following route:

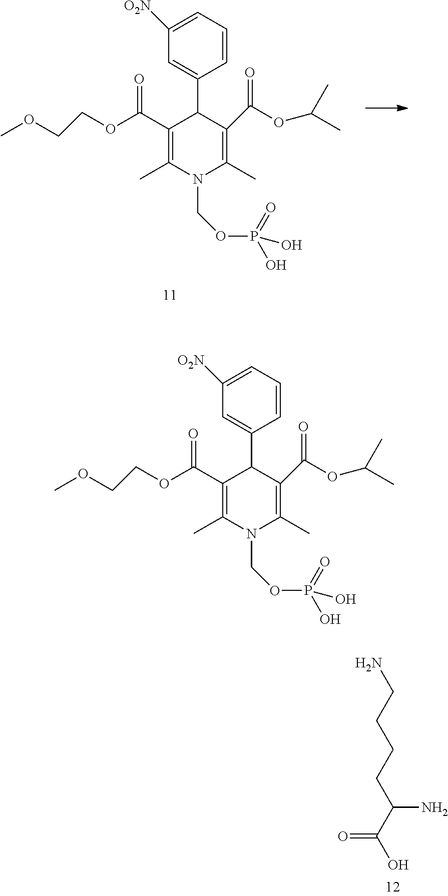

Compound 11 (1 g, 1.4 mmol) was dissolved in 7 mL of ethanol, and 0.7 mL of lysine aqueous solution (1.5 eq) was added dropwise at 40° C. After 0.5 h, 14 mL of ethanol was added, and the reaction was cooled to 0° C. overnight. The reaction mixture was filtered to afford compound 12 as a white solid (533 mg, 56%).

Compound 12 has a solubility of 80 mg/mL in water at room temperature. A portion of compound 12 was throughly mixed with rat anticoagulated plasma and incubated at 37° C. The drug was extracted with acetonitrile at different time points for HPLC analysis. The half-life for converting compound 12 into Nimodipine in blood was determined to be less than 30 mins.

### Example 8 Preparation of Compound 14

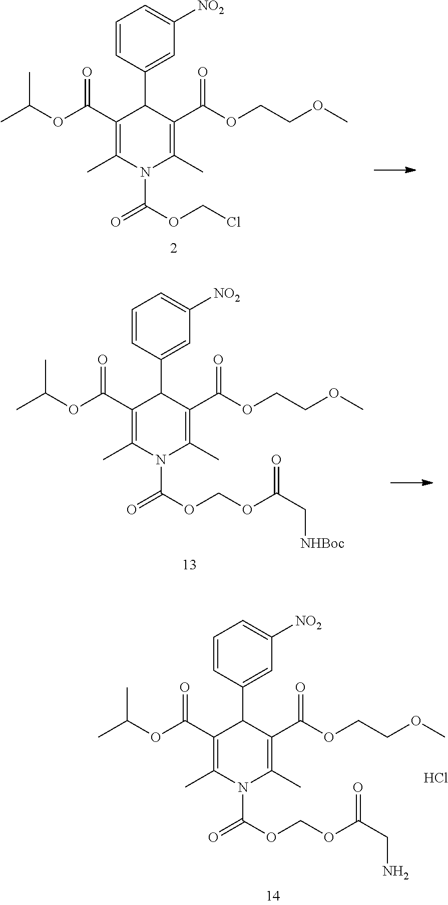

To a solution of potassium N-BOC-glycinate (0.52 g, 2.45 mmol), TBAI (144 mg, 0.39 mmol) and 8 mL of dioxane was added compound 2 (1.0 g, 1.96 mmol), The mixture was stirred at 55-60° C. for 5 hours. The reaction solution was subjected to rotary evaporation and concentrated in vacuo. The concentrate was added with ethyl acetate (40 mL for dissolution), washed with water (15 ml×3), followed by saturated sodium chloride (15 mL), dried over anhydrous sodium sulfate, and concentrated in vacuo. The concentrate was purified by silica-gel column chromatography (petroleum ether/ethyl acetate 3:1) to obtain an intermediate 13 as a light yellow syrup (1.24 g, 97.6%).

The characterization data for the product: ESI-MS, C₃₀H₃₉N₃O₁₃ (649.2), found 672.2 [M+Na]⁺.

Compound 13 (1.12 g) was dissolved in ethyl acetate (4 mL), a solution of HCl in dioxane (5.2 mol/L, 6 mL) was added and the mixture was stirred at room temperature for 5 hours. The reaction solution was concentrated in vacuo, and acetonitrile was added and then concentrated for removal of the residual hydrogen chloride. 20 ml of isopropyl ether was added and the mixture was stirred overnight. Isopropyl ether was poured off, and the solid was dissolved in 15 ml of chloroform and filtered. The filtrate was rotary evaporated to afford compound 14 as a light brown solid (0.64 g, 64%).

The characterization data for the product: ESI-MS: C₂₅H₃₁N₃O₁₁ (549.2) found 550.2 [M+H]⁺. HNMR (500 MHz, CDCl₃) δ 8.67 (brs, 2H), 7.99-7.96 (m, 2H), 7.59 (m, 1H), 7.40-7.36 (m, 1H), 5.92 (s, 2H), 5.55 (brs, 1H), 5.52 (s, 1H), 5.10-5.05 (m, 1H), 4.35-4.26 (m, 2H), 4.08 (s, 2H), 3.61 (m, 2H), 2.53 (s, 3H), 2.51 (s, 3H), 1.29 (d, J=6.2 Hz, 3H).

### Example 9 Preparation of Compound 16

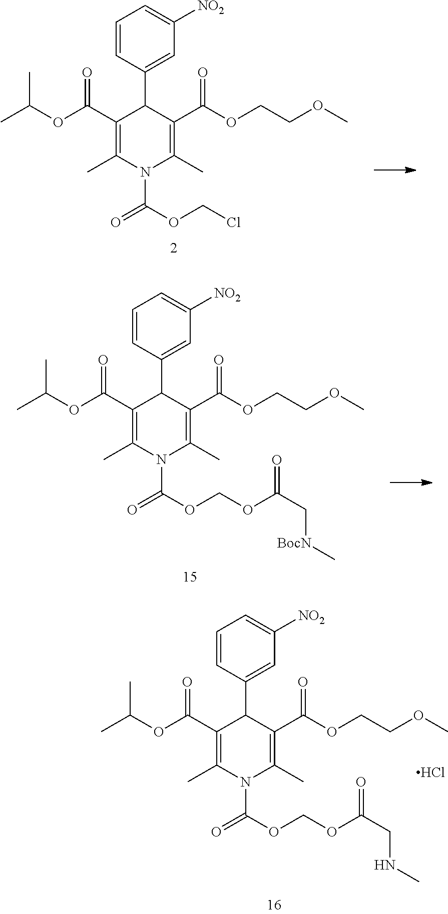

To a solution of potassium N-BOC-sarcosinate (0.56 g, 2.45 mmol), TBAI (144 mg, 0.39 mmol) and 8 mL of dioxane was added compound 2 (1.0 g, 1.96 mmol). The mixture was stirred at 55-60° C. overnight. The reaction solution was subjected to rotary evaporation and concentrated in vacuo. The concentrate was dissolved in ethyl acetate (30 mL), washed with water (15 ml×3), followed by saturated sodium chloride (15 mL×2), dried over anhydrous sodium sulfate, and concentrated in vacuo to afford an intermediate 15 as a light brown syrup (1.24 g, 95.3%).

The characterization data for the product: ESI-MS, C₃₁H₄₁N₃O₁₃ (663.2), found 564.1[M−Boc+H]⁺.

Compound 15 (1.09 g) was dissolved in ethyl acetate (4 mL), a solution of HCl in dioxane (5.2 mol/L, 6 mL) was added and the mixture was stirred at room temperature for 2 hours. The reaction solution was concentrated in vacuo and acetonitrile was added and then concentrated for removal of the residual hydrogen chloride. 20 ml of isopropyl ether was added and the reaction mixture was stirred overnight. Isopropyl ether was poured off, and the solid was dissolved in 15 ml of chloroform, filtered. The filtrate was rotary evaporated to afford compound 16 as a light brown solid (0.65 g, 66.0%).

The characterization data for the product: ESI-MS, C₂₆H₃₃N₃O₁₁ (563.2), found 564.1 [M+H]⁺. ¹H-NMR (500 MHz, CDCl₃) δ 10.06 (brs, 1H), 8.03 (d, J=8.1 Hz, 1H), 7.93 (t, J=1.75 Hz, 1H), 7.65 (d, J=7.9 Hz, 1H), 7.42 (t, J=7.95, 1H), 5.93 (s, 1H), 5.30 (s, 1H), 5.10 (m, 1H), 4.37 (m, 1H), 4.29 (m, 1H), 3.92 (s, 2H), 3.63 (m, 2H), 3.36 (s, 3H), 2.86 (s, 3H), 2.56 (s, 3H), 2.54 (s, 3H), 1.30 (d, J=6.3 Hz, 3H), 1.26 (d, J=6.3 Hz, 3H).

### Example 10 Preparation of Compound 18

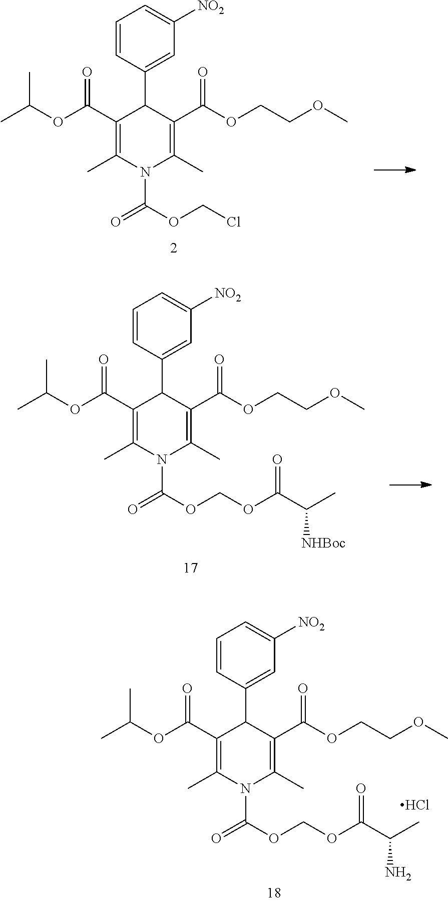

To a solution of potassium BOC-L-alaninate (0.56 g, 2.45 mmol), TBAI (144 mg, 0.39 mmol) and 8 mL of dioxane was added compound 2 (1.0 g, 1.96 mmol). The mixture was stirred at 55-60° C. for 4 hours. The reaction solution was subjected to rotary evaporation and concentrated in vacuo. The concentrate was dissolved in ethyl acetate (30 mL), washed with water (15 ml×2), followed by saturated sodium chloride (15 mL), dried over anhydrous sodium sulfate, filtered, and concentrated in vacuo to afford an intermediate 17 as a light brown syrup (1.25 g, 96.1.0%).

The characterization data for the product: ESI-MS, C₃₁H₄₁N₃O₁₃ (663.2), found 686.2 [M+Na]⁺.

Compound 17 (1.15 g) was dissolved in ethyl acetate (6 mL), a solution of HCl in dioxane (5.2 mol/L, 6 mL) was added and the mixture was stirred at room temperature for 2 hours. The reaction solution was concentrated in vacuo and acetonitrile was added and then concentrated for removal of the residual hydrogen chloride. 15 ml of isopropyl ether was added and the reaction mixture was stirred at 50° C. for 5 mins. Isopropyl ether was poured off, and 15 ml of isopropyl ether was added and the reaction mixture was stirred at 50° C. for another 5 mins. Isopropyl ether was poured off, and the solid was dissolved in 20 ml of chloroform, filtered. The filtrate was rotary evaporated to afford compound 18 as a light brown solid (0.61 g, 58.7%).

The characterization data for the product: ESI-MS, C₂₆H₃₃N₃O₁₁ (563.2), found 564.1 [M+H]⁺. ¹H-NMR (CDCl₃, 500 MHz) δ 8.85 (brs, 2H), 8.00 (m, 1H), 7.97 (s, 1H), 7.59 (d, J=8.0 Hz, 1H), 7.40 (t, J=8.0 Hz, 1H), 5.97 (dd, J=5.8, 1.0 Hz, 1H), 5.87 (dd, J=5.8, 1.0 Hz, 1H), 5.30 (s, 1H), 5.09 (m, 1H), 4.35 (m, 1H), 4.28 (m, 1H), 3.61 (m, 2H), 3.351 (s, 1.5H), 3.349 (s, 1.5H), 2.54 (s, 1.5H), 2.53 (s, 1.5H), 2.53 (s, 1.5H), 2.51 (s, 1.5H), 1.71 (d, J=7.2 Hz, 3H), 1.30 (d, J=6.3 Hz, 3H), 1.26 (m, 3H).

### Example 11 Preparation of Compound 20

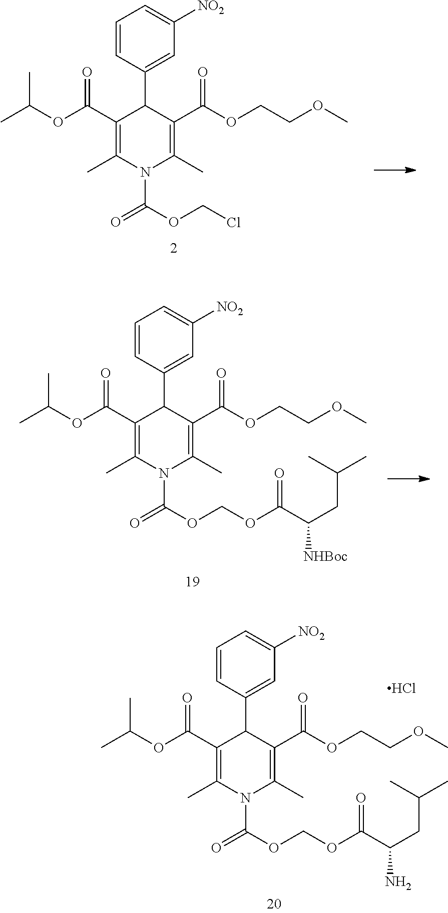

To a solution of potassium BOC-L-leucinate (605 mg, 2.45 mmol), TBAI (144 mg, 0.39 mmol) and 8 mL of dioxane was added compound 2 (1.0 g, 1.96 mmol). The mixture was stirred at 55-60° C. overnight. The reaction solution was subjected to rotary evaporation and concentrated in vacuo. The concentrate was dissolved in ethyl acetate (30 mL), washed with water (15 ml×2), followed by saturated sodium chloride (15 mL), dried over anhydrous sodium sulfate, filtered, and concentrated in vacuo to obtain an intermediate 19 as a light brown syrup (1.5 g, 108.7%).

The characterization data for the product: ESI-MS, C₃₄H₄₇N₃O₁₃ (705.3), found 728.3[M+Na]⁺.

Compound 19 (1.35 g) was dissolved in THF (10 mL), a solution of HCl in dioxane (5.2 mol/L, 5 mL) was added and the mixture was stirred at room temperature for 4 hours. The reaction solution was concentrated in vacuo and acetonitrile was added and then concentrated for removal of the residual hydrogen chloride. 15 ml of isopropyl ether was added and the mixture was stirred at room temperature overnight. The solid was dissolved in 10 ml of chloroform, filtered. The filtrate was rotary evaporated to afford compound 20 as a light brown solid (1.0 g, 81.3%).

The characterization data for the product: ESI-MS, C₂₉H₃₉N₃O₁₁ (605.2), found 606.2 [M+Na]⁺. ¹H-NMR (500 MHz, CDCl₃) δ 8.94 (brs, 2H) 8.02-7.98 (m, 2H), 7.58 (d, J=7.6 Hz, 1H), 7.41 (t, J=7.9 Hz, 1H), 5.97 (d, J=5.9 Hz, 1H), 5.85 (d, J=5.9 Hz, 1H), 5.29 (s, 1H), 5.09 (m, 1H), 4.36 (m, 1H), 4.28 (m, 1H), 3.61 (m, 2H), 3.35 (s, 3H), 4.14 (m, 1H), 2.54 (d, J=1.4 Hz, 3H), 2.52 (d, J=2.15 Hz, 3H), 1.99-1.92 (m, 2H), 1.83 (m, 1H), 1.30 (d, J=6.3 Hz, 3H), 1.26 (d, J=6.3 Hz, 3H), 0.98-0.94 (m, 6H).

### Example 12 Preparation of Compound 21

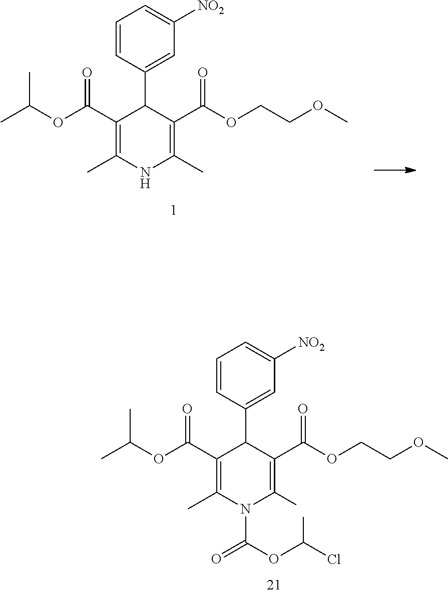

To a solution of Nimodipine (1) (4.5 g, 10.76 mmol) in tetrahydrofuran (17 mL) was added NaH (60%) (0.76 g, 19.0 mmol) at 0° C. The mixture was stirred for 30 minutes, and the reaction was cooled to −40° C. 1-chloroethyl chloroformate (2.07 g, 14.48 mmol) was added dropwise. After the addition in dropwise is completed, the reaction solution was stirred at −40° C. for 20 minutes, and allowed to spontaneously warmed to room temperature and stirred overnight. The reaction solution was concentrated in vacuo, and the residue was dissolved in 60 mL of ethyl acetate, washed with saturated sodium bicarbonate (20 mL×2), followed by saturated sodium chloride (20 mL), dried over anhydrous sodium sulfate, filtered, rotary evaporated, and purified by silica-gel column chromatography (PE/EA 5:1) to afford compound 21 as a light yellow syrup (2.97 g, 52.7%).

The characterization data for the product: ESI-MS, C₂₄H₂₉ClN₂O₉ (524.1), found 525.1[M+H]⁺. ¹H-NMR (500 MHz, CDCl₃) δ 8.05 (dd, J=3.8, 1.8 Hz, 1H), 8.02 (m, 1H), 7.53 (m, 1H), 7.38 (t, J=8.0 Hz, 1H), 6.53 (q, J=5.8 Hz, 1H), 5.30 (s, 1H), 5.12 (m, 1H), 4.38 (m, 1H), 4.31 (m, 1H), 3.63 (m, 2H), 3.37 (s, 1.5H), 3.36 (s, 1.5H), 2.59 (s, 1.5H), 2.57 (s, 1.5H), 2.568 (s, 1.5H), 2.55 (s, 1.5H), 1.83 (d, J=5.8 Hz, 1.5H), 1.82 (d, J=5.8 Hz, 1.5H), 1.32 (d, J=6.3 Hz, 1.5H), 1.317 (d, J=6.3 Hz, 1.5H), 1.30 (d, J=6.3 Hz, 1.5H), 1.26 (d, J=6.3 Hz, 1.5H).

### Example 13 Preparation of Compound 23

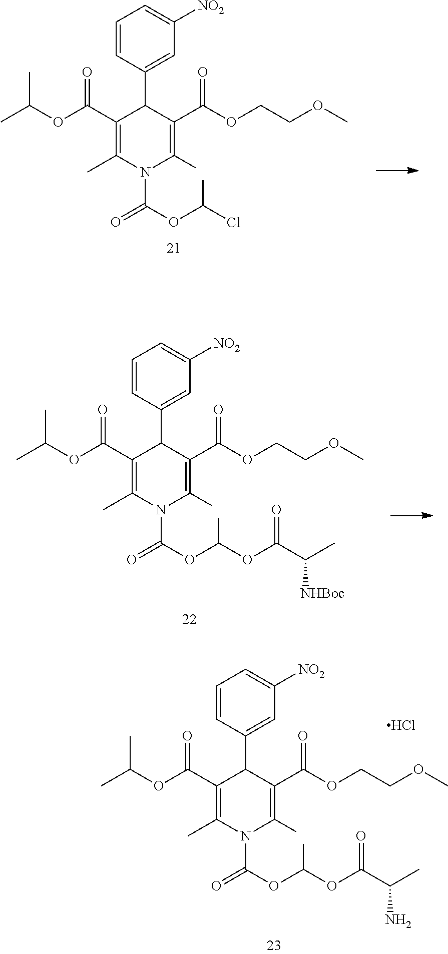

To a solution of potassium BOC-L-alaninate (540 mg, 2.38 mmol), TBAI (144 mg, 0.38 mmol) and 8 mL of dioxane was added compound 21 (1.0 g, 1.9 mmol). The mixture was stirred at 55-60° C. for 4 hours. The reaction solution was subjected to rotary evaporation and concentrated in vacuo. The residue was purified by silica-gel column chromatography (PE/EA 5:1) to obtain an intermediate 22 as a colorless syrup (0.78 g, 60.5%).

The characterization data for the product: ESI-MS, C32H43N3O13 (677.2), found 700.2[M+Na]⁺.

Compound 22 (0.62 g) was dissolved in ethyl acetate (4 mL), a solution of HCl in dioxane (5.2 mol/L, 4 mL) was added and the mixture was stirred at room temperature for 6 hours. The reaction solution was concentrated in vacuo and acetonitrile (10 mL) was added and then concentrated for removal of the residual hydrogen chloride. The concentrate was dissolved in 1.5 mL of THF and precipitated from 20 mL of isopropyl ether. The supernatant was decanted and the solid was treated with THF/isopropyl ether for three times. The resulting precipitate was dissolved in 10 ml of chloroform, filtered. The filtrate was rotary evaporated to afford compound 23 as an off-white foam-like solid (0.416 g, 74.3%).

The characterization data for the product: ESI-Ms, C₂₇H₃₅N₃O₁₁ (577.2), found 578.2[M+H]⁺. ¹H-NMR (CDCl₃, 500 MHz) δ 8.80 (s, 2H), 8.03 (m, 2H), 7.55 (m, 1H), 7.41 (m, 1H), 6.90 (m, 1H), 5.30 (s, 0.5H), 5.29 (s, 0.5H), 5.10 (m, 1H), 4.36 (m, 1H), 4.29 (m, 1H), 4.20 (m, 1H), 3.62 (m, 2H), 3.36 (m, 3H), 2.55-2.47 (m, 6H), 1.71 (d, J=6.4 Hz, 1.5H), 1.60 (d, J=6.4 Hz, 1.5H), 1.53 (m, 3H), 1.29 (m, 6H).

### Example 14 Preparation of Compound 24

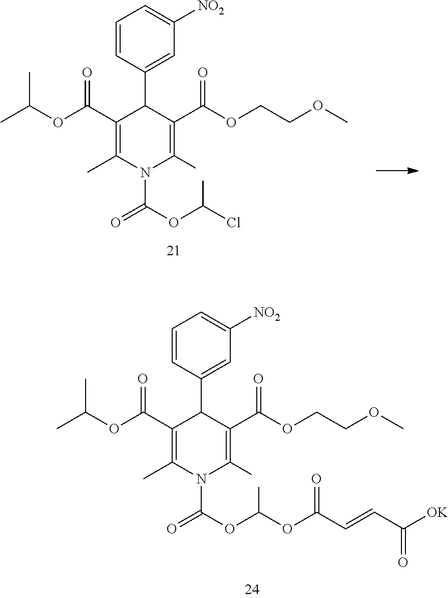

To a solution of fumaric acid (212 mg, 1.82 mmol), DIPEA (234 mg, 1.82 mmol), TBAI (140 mg, 0.38 mmol) and 8 mL of acetonitrile was added compound 21 (0.8 g, 1.52 mmol). The mixture was stirred at 55-60° C. overnight. The reaction solution was subjected to rotary evaporation and concentrated in vacuo. The residue was dissolved in ethyl acetate (50 mL), washed with 0.5 mol/L hydrochloric acid (6 ml×2), followed by saturated sodium chloride (18 mL×5), dried over anhydrous sodium sulfate, filtered, concentrated in vacuo and dissolved in methanol (10 mL). The pH was adjusted to about 7.0 by adding KOH in methanol (40 mg of KOH dissolved in 5 mL of methanol). The solution was rotary evaporated, and the residue was dissolved in 1.5 ml of THE 20 mL of isopropyl ether was added for precipitation. The supernatant was decanted, and the solid was further treated with THF/isopropyl ether for three times. The resulting precipitate was dissolved in 10 ml of chloroform, filtered. The filtrate was rotary evaporated to afford compound 24 as a light brown solid (0.310 g, 31.7%).

The characterization data for the product: ESI-MS, C₂₈H₃₁KN₂O₁₃ (642.1), found 603.2[M−K]⁻. ¹H-NMR (CDCl₃, 500 MHz) δ 8.00 (m, 2H), 7.52 (d, J=7.6 Hz, 1H), 7.37 (td, J=7.8, 1.9 Hz, 1H), 6.85 (dd, J=15.8, 2.5 Hz, 1H) 6.79 (q, J=5.5 Hz, 1H), 6.40 (dd, J=15.8, 1.9 Hz, 1H), 5.27 (s, 1H), 5.08 (m, 1H), 4.34 (m, 1H), 4.27 (m, 1H), 3.60 (m, 2H), 3.33 (s, 1.5H), 3.31 (s, 1.5H), 2.51 (s, 1.5H), 2.50 (s, 1.5H), 2.44 (s, 1.5H), 2.43 (s, 1.5H), 1.40 (m, 3H), 1.27 (m, 6H).

### Example 15 Preparation of Compound 26

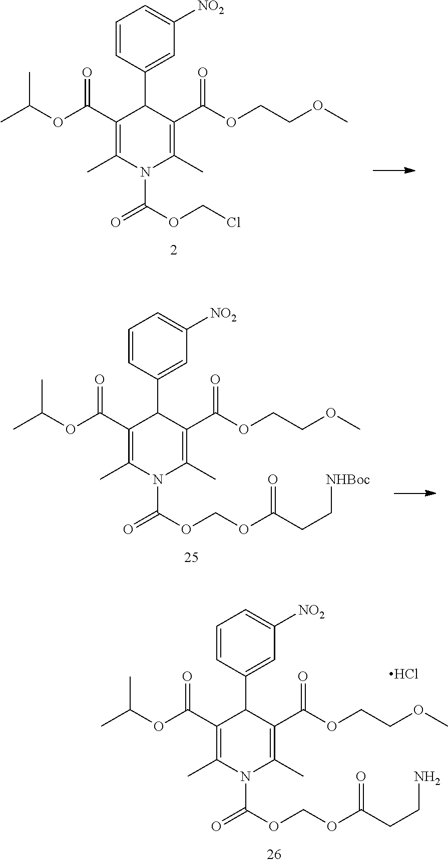

To a solution of potassium BOC-beta-alaninate (0.56 g, 2.45 mmol), TBAI (144 mg, 0.39 mmol) and 8 mL of dioxane was added compound 2 (1.0 g, 1.96 mmol). The mixture was stirred at 55-60° C. overnight. The reaction solution was subjected to rotary evaporation and concentrated in vacuo. The residue was added with ethyl acetate (35 mL for dissolution), washed with water (15 ml×2), followed by saturated sodium chloride (15 mL×2), dried over anhydrous sodium sulfate, and concentrated in vacuo to obtain an intermediate 25 as a light brown syrup (1.36 g, 104.6%).

The characterization data for the product: ESI-MS, C₃₁H₄₁N₃O₁₃ (663.2), found 564.2[M−Boc+H]⁺.

Compound 25 (1.24 g) was dissolved in ethyl acetate (4 mL), a solution of HCl in dioxane (5.2 mol/L, 6 mL) was added and the mixture was stirred at room temperature for 2 hours. The reaction solution was concentrated in vacuo and 15 mL of acetonitrile was added and then concentrated for removal of the residual hydrogen chloride. 20 ml of isopropyl ether was then added and the mixture was stirred overnight. Isopropyl ether was poured off, and the solid was dissolved in 15 ml of chloroform, filtered. The filtrate was rotary evaporated to afford compound 26 as a light brown solid (0.72 g, 64.3%).

The characterization data for the product: ESI-MS, C₂₆H₃₃N₃O₁₁ (563.2), found 564.1 [M+H]⁺. ¹H-NMR (CDCl₃, 500 MHz) δ 8.25 (brs, 2H), 8.02 (m, 1H), 7.97 (s, 1H), 7.59 (d, J=7.8 Hz, 1H), 7.40 (t, J=8.0 Hz, 1H), 5.84 (s, 2H), 5.28 (s, 1H), 5.09 (m, 1H), 4.35 (m, 1H), 4.28 (m, 1H), 3.61 (m, 2H), 3.38 (m, 2H), 3.35 (s, 3H), 3.00 (t, J=6.4 Hz, 2H), 2.53 (s, 3H), 2.51 (s, 3H), 1.29 (d, J=6.3 Hz, 3H), 1.25 (d, J=6.3 Hz, 3H).

### Example 16 Preparation of Compound 28

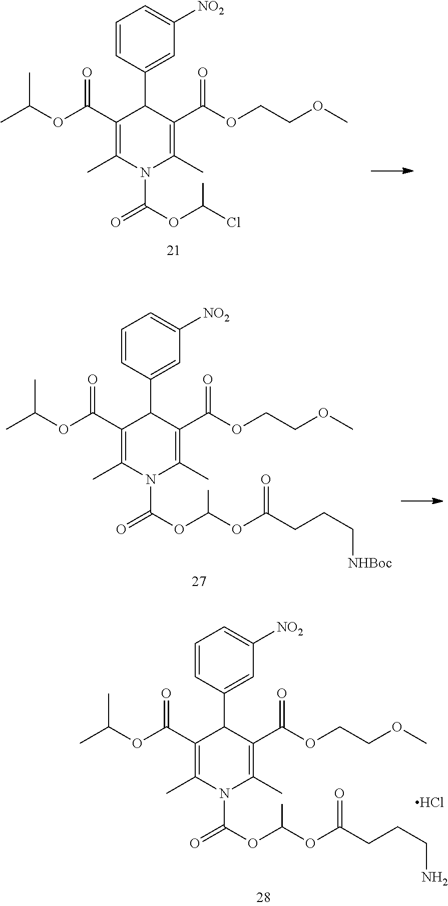

To a solution of potassium BOC-gamma-aminobutyrate (0.287 g, 1.19 mmol), TBAI (70 mg, 0.19 mmol) and 10 mL of dioxane was added compound 21 (0.50 g, 0.95 mmol). The mixture was stirred at 60° C. for 8 hour. The reaction solution was subjected to rotary evaporation and concentrated in vacuo. The residue was dissolved in ethyl acetate (30 mL), washed with saturated NaHCO₃ (10 ml×3), followed by saturated sodium chloride (10 mL×2), dried over anhydrous sodium sulfate, filtered and concentrated in vacuo to obtain an intermediate 27 as a light brown syrup (700 mg, 106.2%).

The characterization data for the product: ESI-MS, C₃₃H₄₅N₃O₁₃ (691.3), found 592.1 [M+Cl]⁻.

To a solution of HCl in dioxane (5.2 mol/L, 8 mL) was added compound 27 (700 mg). The mixture was stirred at room temperature for 5 hours. The reaction solution was concentrated in vacuo. The residue was dissolved in acetonitrile (1.5 mL), and precipitated from 15 ml of isopropyl ether, then the supernatant was poured off and the precipitate was dissolved in acetonitrile, and then precipitated from isopropyl ether, which were repeated five times. The precipitate was dried in vacuo to afford compound 28 as a light brown solid (90 mg, 15.1%).

The characterization data for the product: ESI-MS, C₂₈H₃₇N₃O₁₁ (591.1), found 592.1 [M+H]⁺. ¹H-NMR (CDCl₃, 400 MHz) δ 8.25 (brs, 2H), 8.05 (m, 2H), 7.55 (d, J=7.8 Hz, 1H), 7.41 (t, J=7.8 Hz, 1H), 6.81 (q, J=5.2 Hz, 1H), 5.30 (s, 1H), 5.11 (m, 1H), 4.37 (m, 1H), 4.30 (m, 1H), 3.63 (m, 2H), 3.363 (s, 1.5H), 3.357 (s, 1.5H), 3.11 (m, 2H), 2.56 (s, 1.5H), 2.54 (s, 1.5H), 2.51 (s, 1.5H), 2.48 (s, 1.5H), 2.47 (m, 2H), 2.10 (m, 2H), 1.47 (d, J=5.3 Hz, 3H), 1.31 (d, J=6.2 Hz, 3H), 1.27 (m, 3H).

### Example 17 Preparation of Compound 30

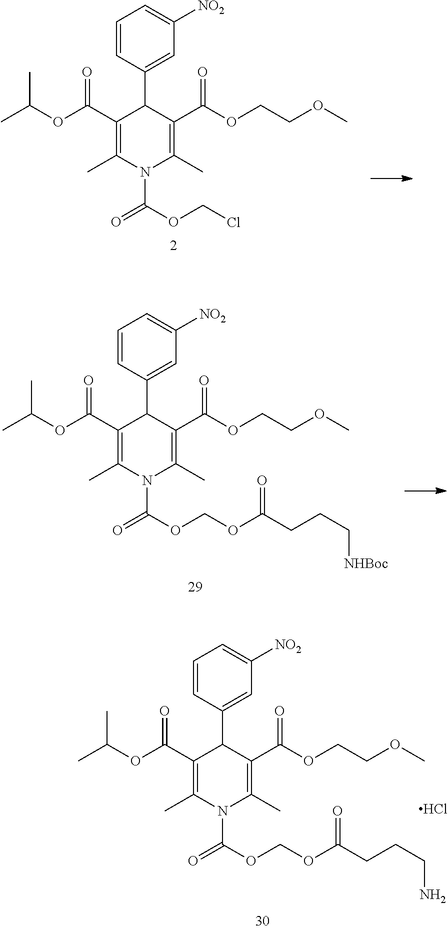

To a solution of potassium BOC-gamma-aminobutyrate (0.301 g, 1.25 mmol), TBAI (74 mg, 0.2 mmol) and 10 mL of dioxane was added compound 2 (0.50 g, 0.98 mmol). The mixture was stirred at 60° C. overnight. The reaction solution was subjected to rotary evaporation and concentrated in vacuo. The residue was dissolved in ethyl acetate (35 mL), washed with saturated NaHCO₃ (10 ml×3), followed by saturated sodium chloride (10 mL×2), dried over anhydrous sodium sulfate, and concentrated in vacuo to obtain an intermediate 29 as a light brown syrup (730 mg, 112.3%).

The characterization data for the product: ESI-MS, C₃₂H₄₃N₃O₁₃ (677.3), found 712.2 [M+Cl]⁻.

Compound 29 (720 mg) was dissolved in tetrahydrofuran (6 mL), a solution of HCl in dioxane solution (5.2 mol/L, 6 mL) was added and the mixture was stirred at room temperature for 3 hours. The reaction solution was concentrated in vacuo, and the residue was dissolved in 15 mL of acetonitrile, filtered. The filtrate was rotary evaporated. The concentrate was dissolved in tetrahydrofuran (1.5 mL), and precipitated from 20 ml of isopropyl ether, then the isopropyl ether was poured off, and the precipitate was dissolved in tetrahydrofuran, and then precipitated from isopropyl ether, which were repeated five times. The precipitate was dried in vacuo to afford compound 30 as a light yellow solid (0.300 g, 64.3%).

The characterization data for the product: ESI-MS, C₂₇H₃₅N₃O₁₁ (577.2), found 578.2 [M+H]⁺. ¹H-NMR (CDCl₃, 400 MHz) δ 8.19 (brs, 2H), 8.01 (m, 2H), 7.59 (d, J=7.8 Hz, 1H), 7.40 (t, J=7.9 Hz, 1H), 5.79 (s, 2H), 5.29 (s, 1H), 5.10 (m, 1H), 4.36 (m, 1H), 4.29 (m, 1H), 3.62 (m, 2H), 3.35 (s, 3H), 3.13 (s, 2H), 2.57 (t, J=6.9 Hz, 2H), 2.54 (s, 3H), 2.52 (s, 3H), 2.13 (t, J=6.9 Hz, 2H), 1.30 (d, J=6.2 Hz, 3H), 1.26 (d, J=6.2 Hz, 3H).

### Example 18 Preparation of Compound 32

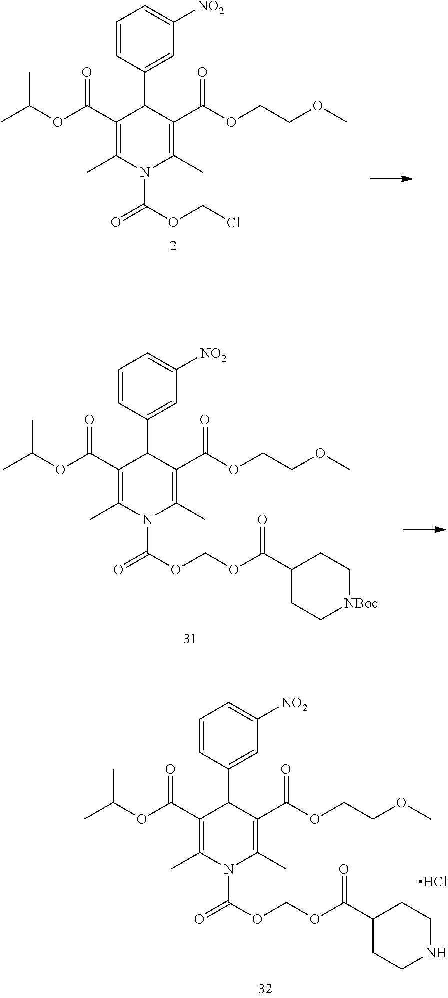

To a solution of potassium BOC-4-piperidine formate (0.334 g, 1.25 mmol), TBAI (74 mg, 0.2 mmol) and 10 mL of dioxane was added compound 2 (0.50 g, 0.98 mmol). The mixture was stirred at 60° C. overnight. The reaction solution was subjected to rotary evaporation and concentrated in vacuo. The residue was dissolved in ethyl acetate (40 mL), washed with saturated NaHCO₃ (15 ml×3), followed by saturated sodium chloride (15 mL×2), dried over anhydrous sodium sulfate, and concentrated in vacuo to obtain an intermediate 31 as light brown syrup (750 mg, 109.0%).

To a solution of HCl in dioxane (5.2 mol/L, 8 mL) was added compound 31 (750 mg), and the mixture was stirred at room temperature for 4 hours. The reaction solution was concentrated in vacuo. The residue was dissoloved in acetonitrile (1.5 mL), and precipitated from 20 ml of isopropyl ether, then the supernatant was poured off and the precipitate was dissolved in acetonitrile, and then precipitated from isopropyl ether, which were repeated five times. The precipitate was dried in vacuo to afford compound 32 as a light brown solid (0.320 g, 54.3%).

The characterization data for the product: ESI-MS, C₂₉H₃₇N₃O₁₁ (603.2), found 604.2 [M+H]⁺. ¹H-NMR (CDCl₃, 400 MHz) δ 9.66 (brs, 1H), 9.44 (brs, 1H), 8.04 (m, 1H), 7.97 (t, J=1.92 Hz, 1H), 7.63 (d, J=7.8 Hz, 1H), 7.41 (t, J=7.9 Hz, 1H), 5.82 (s, 2H), 5.30 (s, 1H), 5.11 (m, 1H), 4.38 (m, 1H), 4.30 (m, 1H), 3.63 (m, 2H), 3.43 (m, 2H), 3.36 (s, 3H), 3.05 (m, 2H), 2.65 (m, 1H), 2.54 (s, 3H), 2.52 (s, 3H), 2.19 (m, 2H), 2.10 (m, 2H), 1.31 (d, J=6.2 Hz, 3H), 1.27 (d, J=6.3 Hz, 3H).

### Example 19 Preparation of Compound 34

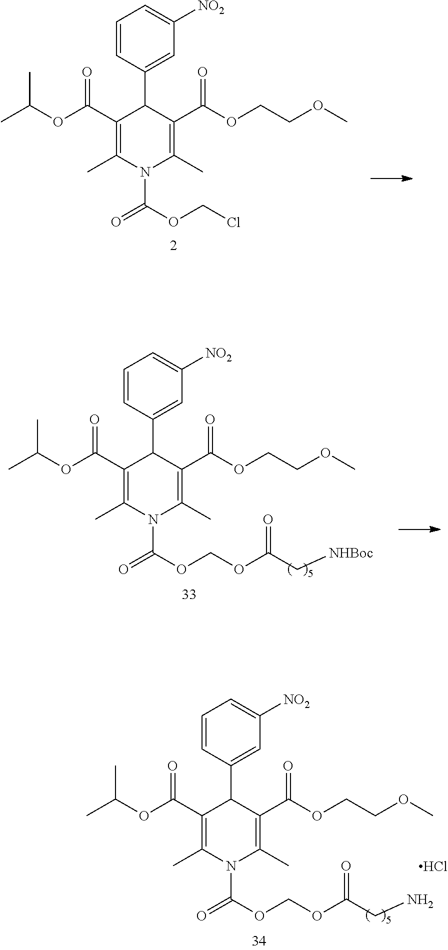

To a solution of potassium BOC-6-aminocaproate (0.337 g, 1.25 mmol), TBAI (74 mg, 0.2 mmol) and 10 mL of dioxane was added compound 2 (0.50 g, 0.98 mmol). The mixture was stirred at 60° C. for 8 hours and then rotary evaporated for removal of the solvent. The concentrate was dissolved in ethyl acetate (40 mL), washed with saturated NaHCO₃ (10 ml×3), followed by saturated sodium chloride (10 mL×2), dried over anhydrous sodium sulfate, and concentrated in vacuo to obtain an intermediate 33 as a light brown syrup (670 mg, 97.0%).

The characterization data for the product: ESI-MS, C₃₄H₄₇N₃O₁₃ (705.3), found 740.3 [M+Cl]⁻.

To a solution of HCl in dioxane (5.2 mol/L, 8 mL) was added compound 33 (665 mg) and the mixture was stirred at room temperature for 4 hours. The reaction solution was concentrated in vacuo and then 10 mL of acetonitrile was added and then concentrated for removal of the hydrogen chloride remained in the second steps. The concentrate was dissolved in acetonitrile (1.5 mL), and precipitated from isopropyl ether (20 ml), then the supernatant was poured off and the precipitate was dissolved in acetonitrile, and then precipitated from isopropyl ether, where were repeated five times. The precipitate was dissolved in acetonitrile (2 mL), filtered, and concentrated to afford compound 34 as a light brown solid (0.16 mg, 27.0%).

The characterization data for the product: ESI-MS, C₂₉H₃₉N₃O₁₁ (605.3), found 606.1 [M+H]⁺. ¹H-NMR (CDCl₃, 400 MHz) δ 8.23 (brs, 2H), 8.04 (m, 2H), 7.58 (d, J=7.7 Hz, 1H), 7.40 (t, J=7.7 Hz, 1H), 5.79 (s, 2H), 5.29 (s, 1H), 5.11 (m, 1H), 4.37 (m, 1H), 4.30 (m, 1H), 3.63 (t, J=4.6 Hz 2H), 3.36 (s, 3H), 3.01 (m, 2H), 2.55 (s, 3H), 2.52 (s, 3H), 2.37 (t, J=7.2 Hz, 2H), 1.81 (m, 2H), 1.66 (m, 2H), 1.45 (m, 2H), 1.31 (d, J=6.2 Hz, 3H), 1.27 (d, J=6.3 Hz, 3H).

### Example 20 Preparation of Compound 36

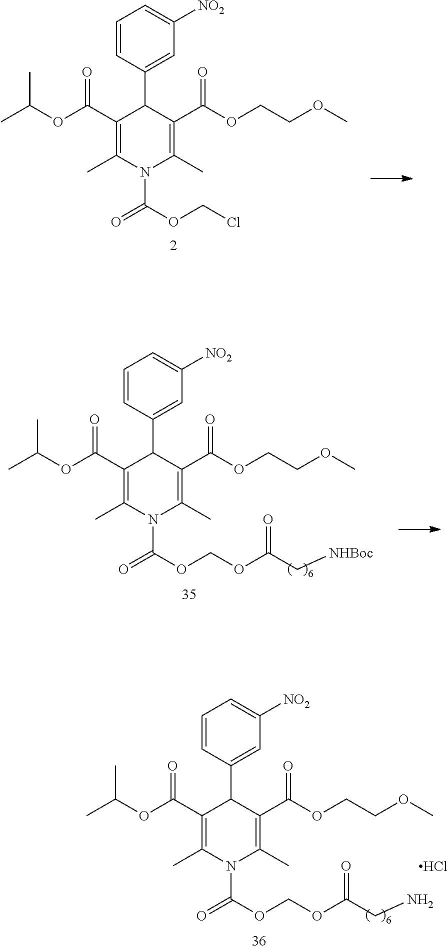

To a solution of potassium BOC-7-aminoheptanoate (0.354 g, 1.25 mmol), TBAI (74 mg, 0.2 mmol) and 10 mL of dioxane was added compound 2 (0.50 g, 0.98 mmol). The mixture was stirred at 60° C. for 8 hours, and then rotary evaporated for removal of the solvent. The concentrate was dissolved in ethyl acetate (40 mL), washed with saturated NaHCO₃ (10 ml×3), followed by saturated sodium chloride (10 mL×2), dried over anhydrous sodium sulfate, and concentrated in vacuo to obtain an intermediate 35 as a light brown syrup (710 mg, 100.0%).

The characterization data for the product: ESI-MS, C₃₅H₄₉N₃O₁₃ (719.3), found 736.4 [M+OH]⁻.

To a solution of HCl in dioxane (5.2 mol/L, 8 mL) was added compound 35 (705 mg) and the mixture was stirred at room temperature for 5 hours. The reaction solution was concentrated in vacuo and acetonitrile (10 mL) was added and then concentrated for removal of the hydrogen chloride remained in the second steps. The concentrate was dissolved in acetonitrile (1.5 mL), and precipitated from isopropyl ether (20 ml), then the supernatant was poured off and the precipitate was dissolved in acetonitrile and then precipitated from isopropyl ethe, which were repeated five times. The precipitate was dissolved in acetonitrile (2 mL), filtered, and concentrated to afford compound 36 as a light brown solid (0.329 mg, 54.3%).

The characterization data for the product: ESI-MS, C₃₀H₄₁N₃O₁₁ (619.3), found 620.1 [M+H]⁺. HNMR (CDCl₃, 400 MHz) δ 8.22 (brs, 2H), 8.04 (m, 2H), 7.58 (d, J=7.7 Hz, 1H), 7.40 (t, J=7.7 Hz, 1H), 5.79 (s, 2H), 5.29 (s, 1H), 5.11 (m, 1H), 4.37 (m, 1H), 4.30 (m, 1H), 3.63 (t, J=4.8 Hz 2H), 3.36 (s, 3H), 3.00 (m, 2H), 2.55 (s, 3H), 2.52 (s, 3H), 2.34 (m, 2H), 1.78 (m, 2H), 1.63 (m, 2H), 1.39 (m, 2H), 1.31 (d, J=6.2 Hz, 3H), 1.27 (d, J=6.3 Hz, 3H).

### Example 21 Preparation of Compound 38

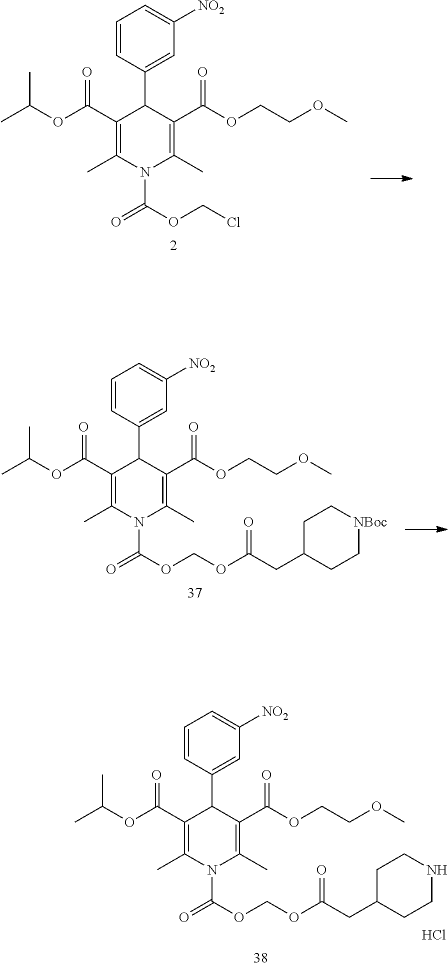

To a solution of potassium BOC-6-piperidineacetate (0.352 g, 1.25 mmol), TBAI (74 mg, 0.2 mmol) and 10 mL of dioxane was added compound 2 (0.50 g, 0.98 mmol). The mixture was stirred at 60° C. for 7 hours, and then rotary evaporated for removal of the solvent. The residue was dissolved in ethyl acetate (40 mL), washed with saturated NaHCO₃ (10 ml×3), followed by saturated sodium chloride (10 mL×2), dried over anhydrous sodium sulfate, and concentrated in vacuo to obtain an intermediate 37 as a light brown syrup (785 mg, 111.8%).

The characterization data for the product: ESI-MS, C₃₅H₄₇N₃O₁₃ (717.3), found 618.1 [M−Boc+H]⁺.

To a solution of HCl in dioxane (5.2 mol/L, 8 mL) was added compound 37 (780 mg) and the mixture was stirred at room temperature for 5 hours. The reaction solution was concentrated in vacuo and acetonitrile (10 mL) was added and then concentrated for removal of the hydrogen chloride remained in the second steps. The concentrate was dissolved in acetonitrile (1.5 mL), and precipitated from isopropyl ether (20 ml), then the supernatant was poured off and the precipitate was dissolved in acetonitrile and then precipitated from isopropyl ether, which were repeated five times. The precipitate was dissolved in acetonitrile (2 mL), filtered, and concentrated to afford compound 38 as a light brown solid (0.500 g, 78.1%).

The characterization data for the product: ESI-MS, C₃₀H₃₉N₃O₁₁ (617.3), found 618.1 [M+H]⁺. ¹H-NMR (CDCl₃, 400 MHz) δ 9.61 (brs, 2H), 8.04 (m, 1H), 7.99 (m, 1H), 7.60 (d, J=7.7 Hz, 1H), 7.41 (t, J=7.7 Hz, 1H), 5.80 (s, 2H), 5.30 (s, 1H), 5.11 (m, 1H), 4.37 (m, 1H), 4.30 (m, 1H), 3.62 (m, 2H), 3.50 (m, 2H), 3.36 (s, 3H), 2.88 (m, 2H), 2.54 (s, 3H), 2.52 (s, 3H), 2.35 (d, J=6.9 Hz, 2H), 2.27 (m, 1H), 1.96 (m, 2H), 1.72 (m, 2H), 1.31 (d, J=6.2 Hz, 3H), 1.27 (d, J=6.3 Hz, 3H).

### Example 22 Preparation of Compound 39

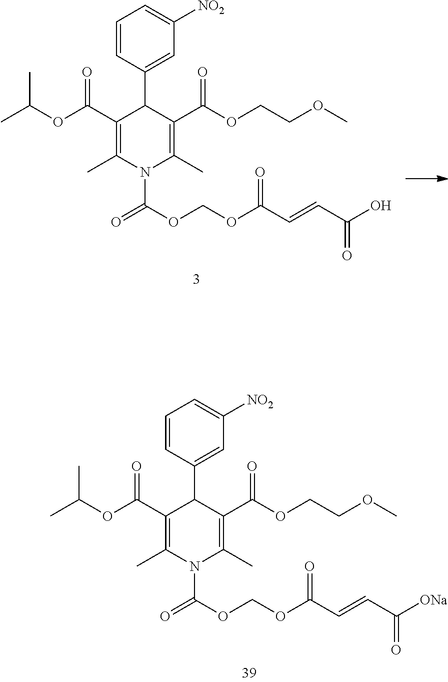

Compound 3 (350 mg) was dissolved in 5 mL of methanol, and the pH was carefully adjusted to approximately 7.0 with 0.5 mol/L of a solution of sodium hydroxide in methanol. The solvent was rotary evaporated. The concentrate was dissolved in 1.5 mL of THF, and precipitated from 15 mL of isopropyl ether. The supernatant was decanted and the solid was treated with THF/isopropyl ether for three times. The resulting precipitate was dissolved in 10 mL of chloroform, filtered. The filtrate was rotary evaporated to afford compound 39 as a light brown solid (0.250 g, 68.9%).

The characterization data for the product: ¹H-NMR (500 MHz, CDCl₃) δ 7.96 (m, 2H), 7.54 (d, J=7.8 Hz, 1H), 7.36 (t, J=7.8 Hz, 1H), 6.90 (d, J=15.8 Hz, 1H) 6.44 (q, J=5.5 Hz, 1H), 5.77 (t, J=6.8 Hz, 2H), 5.26 (s, 1H), 5.07 (m, 1H), 4.33 (m, 1H), 4.26 (m, 1H), 3.59 (t, J=5.0 Hz, 2H), 3.32 (s, 3H), 2.49 (s, 3H), 2.47 (s, 3H), 1.27 (d, J=6.2 Hz, 3H), 1.23 (d, J=6.2 Hz, 3H).

### Example 23 Preparation of Compound 41

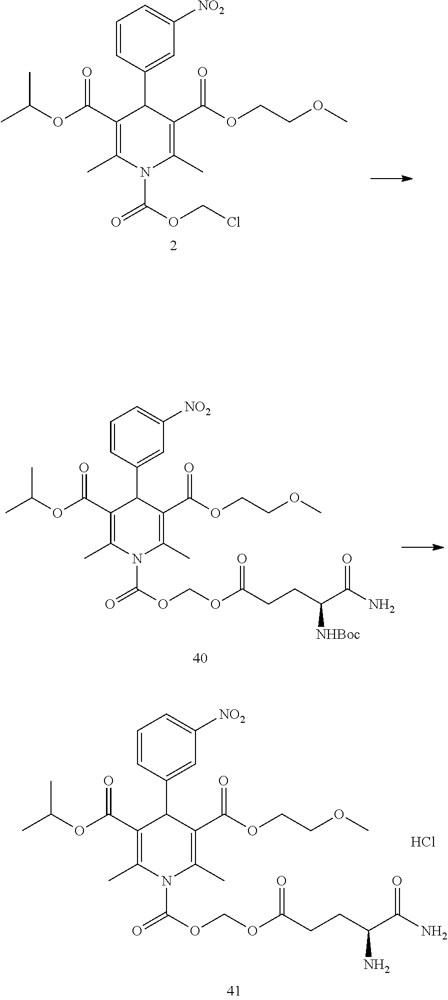

To a solution of potassium BOC-L-isoglutamate (0.355 g, 1.25 mmol), TBAI (74 mg, 0.2 mmol) and 10 mL of dioxane was added compound 2 (0.50 g, 0.98 mmol) The mixture was stirred at 60° C. overnight, and then rotary evaporated for removal of the solvent. The residue was dissolved in ethyl acetate (40 mL), washed with saturated NaHCO₃ (15 mL×3), followed by saturated sodium chloride (15 mL×2), dried over anhydrous sodium sulfate, and concentrated in vacuo to obtain an intermediate 40 as a light brown syrup (650 mg, 92.2%).

The characterization data for the product: ESI-MS, C₃₃H₄₄N₄O₁₄ (720.3), found 621.3 [M−Boc+H]⁺.

To a solution of HCl in dioxane solution (5.2 mol/L, 8 mL) was added compound 40 (630 mg) and the mixture was stirred overnight at room temperature. The reaction solution was concentrated in vacuo and then acetonitrile (15 mL) was added and then concentrated for removal of the hydrogen chloride remained in the second steps. The concentrate was dissolved in acetonitrile (1.5 mL), and precipitated from isopropyl ether (20 ml), then the supernatant was poured off and the precipitate was dissolved in acetonitrile, and then precipitated from isopropyl ether, which were repeated five times. The precipitate was dissolved in acetonitrile (2 mL), filtered, and concentrated to afford compound 41 as a light brown solid (100 mg, 15.6%).

The characterization data for the product: ESI-MS, C₂₈H₃₇ClN₄O₁₂ (656.2), found 621.2 [M−Cl]⁺.

### EXAMPLE

Solubility of Nimodipine derivatives in water and release rate thereof into plasma

1. Solubility in Water

The compound sample prepared according to the above examples was accurately weighed in an appropriate amount, and pure water was added dropwise with a microsyringe while shaking until the solution became clear. The amounts of the sample and pure water were recorded and converted into mg/mL, as the solubility of the sample. The result was shown in the following table, and the original drug, i.e. Nimodipine, was used as a control.

2. Release Rate into Plasma

The compound sample prepared according to the above examples was dissolved in brine in an appropriate amount to formulate into a stock solution between 0.3 and 0.4 mg/mL. 20 μL of stock solution was added into the rat anticoagulated plasma previously incubated at 37° C. for 2 mins, which were throughly mixed, and incubated at 37° C. 100 μL of samples were taken at different time points, and an equal amount of acetonitrile was added to precipitate proteins, then centrifugated and the supernatant was sampled for HPLC analysis. The half-life t_{1/2} (min) was calculated and the results were shown in table 1.

TABLE 1
Solubility of Nimodipine Derivatives in Water and Release Rate thereof into Plasma

| | | | | release rate |
|---|---|---|---|---|
| | | | solubility | into plasma |
| Example No. | Compound No. | structure | (mg/mL) | t_{1/2}(min) |
| 2 | 6 | 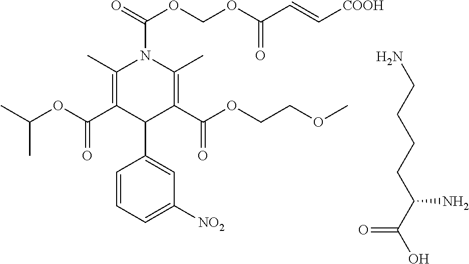 [SMILES: COCCOC(=O)C1=C(C)N(C(=O)OCOC(=O)/C=C/C(=O)O)C(C)=C(C(=O)OC(C)C)C1c1cccc([N+](=O)[O-])c1 ; NCCCC[C@H](N)C(=O)O] | >50 | about 90 |
| 4 | 8 | 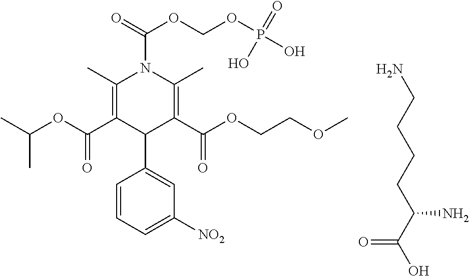 [SMILES: COCCOC(=O)C1=C(C)N(C(=O)OCOP(=O)(O)O)C(C)=C(C(=O)OC(C)C)C1c1cccc([N+](=O)[O-])c1 ; NCCCC[C@H](N)C(=O)O] | >50 | <90 |
| 5 | 9 | 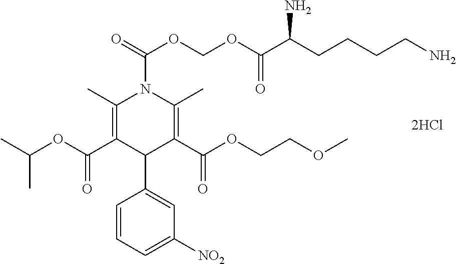 [SMILES: COCCOC(=O)C1=C(C)N(C(=O)OCOC(=O)[C@@H](N)CCCCN)C(C)=C(C(=O)OC(C)C)C1c1cccc([N+](=O)[O-])c1] | >50 | about 150 |
| 7 | 12 | 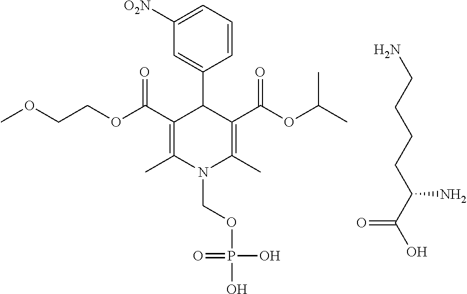 [SMILES: COCCOC(=O)C1=C(C)N(COP(=O)(O)O)C(C)=C(C(=O)OC(C)C)C1c1cccc([N+](=O)[O-])c1 ; NCCCC[C@H](N)C(=O)O] | >50 | <30 |
| 8 | 14 | 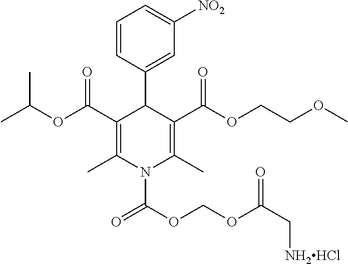 | >50 | <30 |
| 9 | 16 | 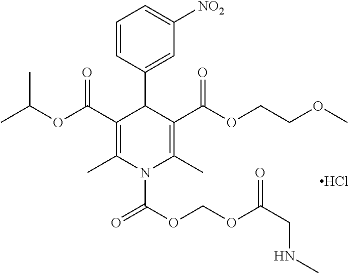 [SMILES: CNCC(=O)OCOC(=O)N1C(C)=C(C(=O)OCCOC)C(c2cccc([N+](=O)[O-])c2)C(C(=O)OC(C)C)=C1C ; Cl] | >50 | <30 |
| 10 | 18 | 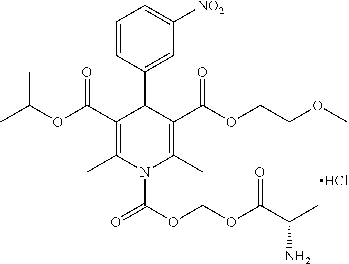 [SMILES: COCCOC(=O)C1=C(C)N(C(=O)OCOC(=O)[C@H](C)N)C(C)=C(C(=O)OC(C)C)C1c1cccc([N+](=O)[O-])c1 ; Cl] | >50 | <30 |
| 11 | 20 | 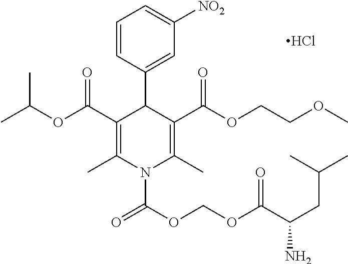 [SMILES: COCCOC(=O)C1=C(C)N(C(=O)OCOC(=O)[C@@H](N)CC(C)C)C(C)=C(C(=O)OC(C)C)C1c1cccc([N+](=O)[O-])c1 ; Cl] | >50 | <30 |
| 13 | 23 | 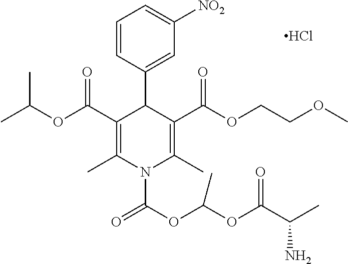 [SMILES: COCCOC(=O)C1=C(C)N(C(=O)OC(C)OC(=O)[C@H](C)N)C(C)=C(C(=O)OC(C)C)C1c1cccc([N+](=O)[O-])c1 ; Cl] | >50 | <30 |
| 14 | 24 | 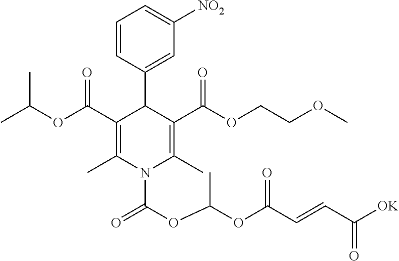 | >50 | <60 |
| 15 | 26 | 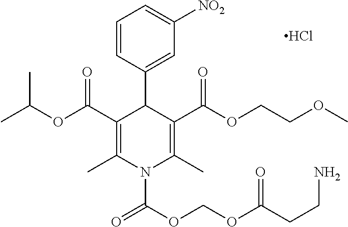 [SMILES: COCCOC(=O)C1=C(C)N(C(=O)OCOC(=O)CCN)C(C)=C(C(=O)OC(C)C)C1c1cccc([N+](=O)[O-])c1 ; Cl] | >50 | <30 |
| 16 | 28 | 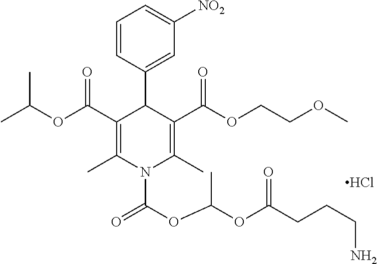 [SMILES: COCCOC(=O)C1=C(C)N(C(=O)OC(C)OC(=O)CCCN)C(C)=C(C(=O)OC(C)C)C1c1cccc([N+](=O)[O-])c1 ; Cl] | >50 | <30 |
| 17 | 30 | 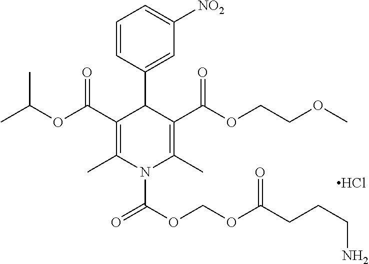 [SMILES: COCCOC(=O)C1=C(C)N(C(=O)OCOC(=O)CCCN)C(C)=C(C(=O)OC(C)C)C1c1cccc([N+](=O)[O-])c1 ; Cl] | >50 | <30 |
| 18 | 32 | 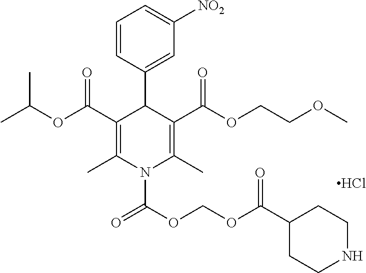 [SMILES: COCCOC(=O)C1=C(C)N(C(=O)OCOC(=O)C2CCNCC2)C(C)=C(C(=O)OC(C)C)C1c1cccc([N+](=O)[O-])c1 ; Cl] | >50 | <30 |
| 19 | 34 | 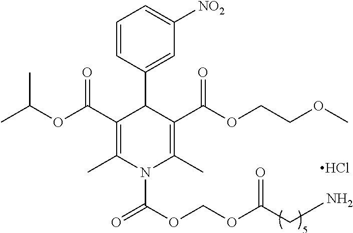 | >50 | <30 |
| 20 | 36 | 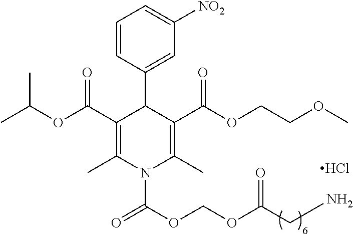 | >50 | <30 |
| 21 | 38 | 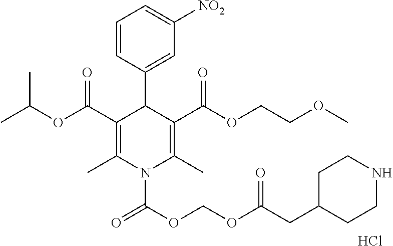 [SMILES: COCCOC(=O)C1=C(C)N(C(=O)OCOC(=O)CC2CCNCC2)C(C)=C(C(=O)OC(C)C)C1c1cccc([N+](=O)[O-])c1 ; Cl] | >50 | <30 |
| 22 | 39 | 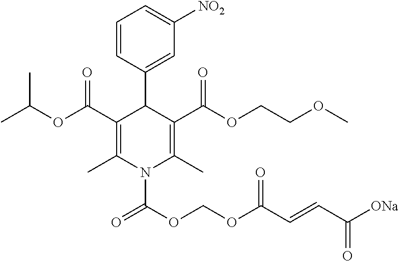 | >50 | <60 |
| 23 | 41 | 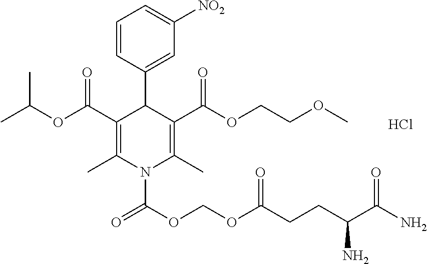 [SMILES: COCCOC(=O)C1=C(C)N(C(=O)OCOC(=O)CC[C@H](N)C(N)=O)C(C)=C(C(=O)OC(C)C)C1c1cccc([N+](=O)[O-])c1 ; Cl] | >50 | <30 |
| Control | Nimodipine | 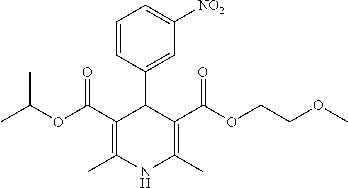 [SMILES: COCCOC(=O)C1=C(C)NC(C)=C(C(=O)OC(C)C)C1c1cccc([N+](=O)[O-])c1] | 0.003 | / |

As can be seen from the above table, Nimodipine derivatives described above have an excellent solubility in water and can be quickly converted into Nimodipine in plasma.

The foregoing embodiments merely represent several embodiments of the present disclosure, which are described in particular and details, but should not be understood as being limited to the scope of the present disclosure. It should be noted that, for those skilled in the art, several variations and improvements may be made without departing from the concept of the present disclosure, and these are all within the protection scope of the present disclosure. Therefore, the protection scope of the present disclosure shall be subject to the appended claims.

1. A water-soluble Nimodipine derivative having a structural characteristic of formula I or a pharmaceutically acceptable salt thereof: 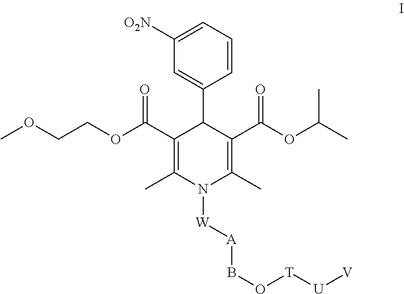
wherein:
W is selected from C═O, C═S, or SO₂; or W is absent;
A is selected from O or S; or A is absent;
B is C(R₄)(R₅), or absent;
each of R₄ and R₅ is independently selected from hydrogen, deuterium, C₁-C₃ alkyl, C₁-C₃ alkyl substituted by R₁₅, aryl, or aryl substituted by R₁₅, and R₄ and R₅ together with the atom to which they are attached can form a 4 to 6-membered ring;
R₁₅ is selected from O, carboxyl, or amino;
T is selected from C═O, SO₂, SO₃R₆, PO₃R₇R₈, or PO₂R₁₇(NHR₁₈); or T is absent;
each of R₆, R₇, and R₈ is independently selected from H, a metal ion, or an ammonium ion;
R₁₇ is selected from aryl, substituted aryl, naphthyl or substituted naphthyl;
NHR₁₈ is an amino acid group;
U is selected from C₁-C₈ alkyl, carboxyl-containing C₁-C₈ alkyl, C₃-C₈ cycloalkyl, aryl, alkenyl, alkynyl, nitrogen-containing heterocycloalkyl, guanidyl-containing C₁-C₈ alkyl, amide-containing C₁-C₈ alkyl, 2-4 peptide alkyl, C₁-C₈ alkyl substituted by R₁₆, C₃-C₈ cycloalkyl substituted by R₁₅, aryl substituted by R₁₅, alkenyl substituted by R₁₅, or alkynyl substituted by R₁₅; or U is absent;
R₁₆ is selected from amino, carboxyl, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;
V is selected from NR₉R₁₀, COOR₁₁, PO₃R₁₂R₁₃ or SO₃R₁₄; or V is absent;
each of R₉ and R₁₀ is independently selected from hydrogen, C₁-C₈ alkyl, or C₁-C₈ alkyl substituted by R₁₅, and R₉ and R₁₀ together with the atom to which they are attached can form a 4 to 8-membered ring;
each of R₁₁, R₁₂, R₁₃, and R₁₄ is independently selected from H, a metal cation, or an ammonium ion; and
the metal cation is selected from sodium ion, potassium ion, lithium ion, calcium ion, or magnesium ion.

2. The water-soluble Nimodipine derivative or the pharmaceutically acceptable salt thereof according to claim 1, wherein the water-soluble Nimodipine derivative is selected from a structure represented by the following formula II: 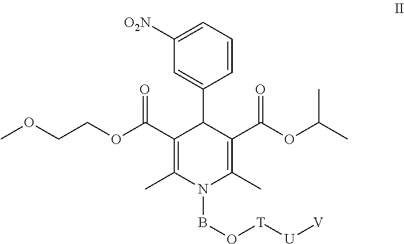
B is C(R₄)(R₅); and
each of R₄ and R₅ is independently selected from hydrogen, deuterium, or C₁-C₃ alkyl.

3. The water-soluble Nimodipine derivative or the pharmaceutically acceptable salt thereof according to claim 1, wherein the water-soluble Nimodipine derivative is selected from a structure represented by the following formula III: 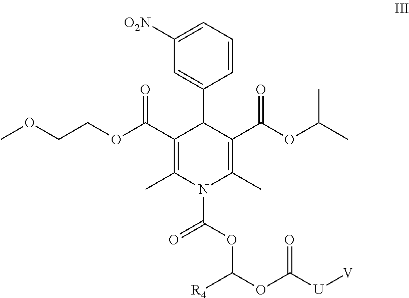
R₄ is selected from hydrogen, deuterium, or C₁-C₃ alkyl.

4. The water-soluble Nimodipine derivative or the pharmaceutically acceptable salt thereof according to claim 3, wherein U is selected from C₁-C₈ alkyl, alkenyl, or C₁-C₈ alkyl substituted by R₁₆;
R₁₆ is selected from amino, or carboxyl;
V is selected from NR₉R₁₀, or COOR₁₁; or V is absent; and
each of R₉ and R₁₀ is independently selected from hydrogen, or C₁-C₈ alkyl.

5. The water-soluble Nimodipine derivative or the pharmaceutically acceptable salt thereof according to claim 1,
wherein U is selected from C₁-C₈ alkyl, alkenyl, nitrogen-containing heterocycloalkyl, guanidyl-containing C₁-C₈ alkyl, amide-containing C₁-C₈ alkyl, 2-4 peptide alkyl, or C₁-C₈ alkyl substituted by R₁₆; or U is absent;
R₁₆ is selected from amino, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;
V is selected from NR₉R₁₀, COOR₁₁ or PO₃R₁₂R₁₃; or V is absent; and
each of R₉ and R₁₀ is independently selected from hydrogen, or C₁-C₈ alkyl.

6. The water-soluble Nimodipine derivative or the pharmaceutically acceptable salt thereof according to claim 4, wherein U together with V can form one of the following groups:

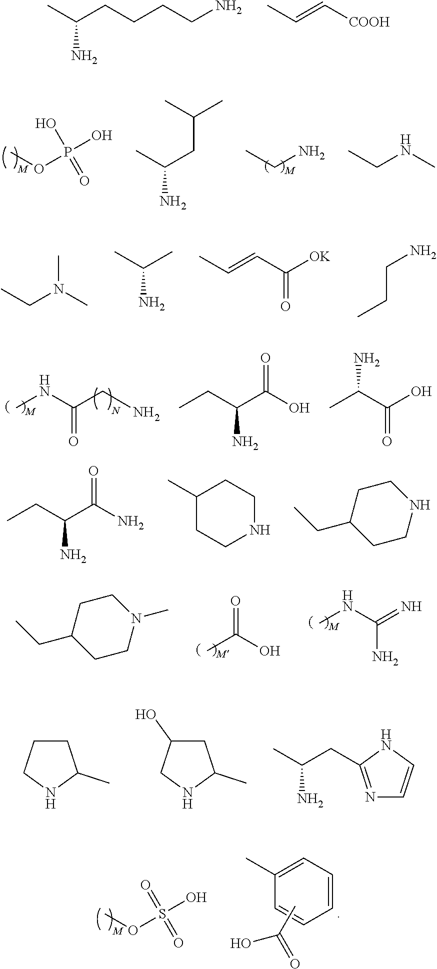

7. The water-soluble Nimodipine derivative or the pharmaceutically acceptable salt thereof according to claim 1, wherein the water-soluble Nimodipine derivative is selected from a structure represented by the following formula IV: 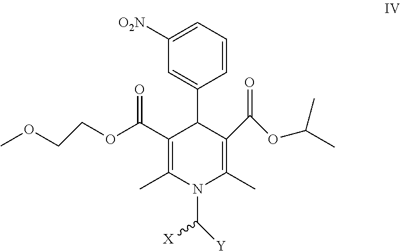
wherein:
when X is H, Y is selected from OH 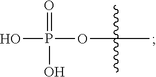 [SMILES: *OP(=O)(O)O]
when X is ═O, Y is selected from 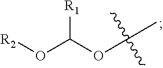
R₁ is selected from hydrogen, C₁-C₆ alkyl, or substituted C₁-C₆ alkyl;
R₂ is selected from one of the following groups: 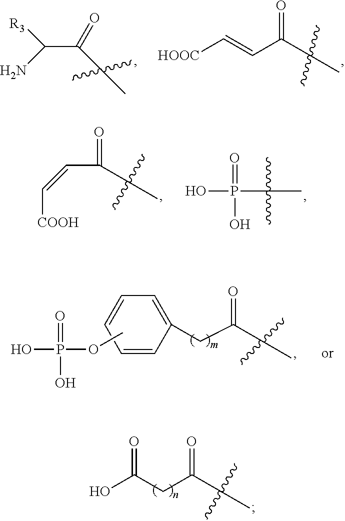
R₃ is selected from hydrogen, C₁-C₆ alkyl, substituted C₁-C₆ alkyl, C₃-C₇ cycloalkyl, substituted C₃-C₇ cycloalkyl, aryl, substituted aryl, heterocyclyl containing O, N, or S heteroatom, substituted heterocyclyl containing O, N, or S heteroatom, heteroaryl containing O, N, or S heteroatom, substituted heteroaryl containing O, N, or S heteroatom, or a side chain group of a natural amino acid;
m is selected from 0, 1, 2, or 3; and
n is selected from 0, 1, or 2.

8. The water-soluble Nimodipine derivative or the pharmaceutically acceptable salt thereof according to claim 7, wherein the natural amino acid is selected from lysine, arginine, or histidine.

9. The water-soluble Nimodipine derivative or the pharmaceutically acceptable salt thereof according to claim 7, wherein the pharmaceutically acceptable salt is selected from sodium salt, potassium salt, calcium salt, magnesium salt, lithium salt, lysine salt, arginine salt, aspartic acid, glutamic acid, tromethamine salt, ethanolamine salt, hydrochloride, sulfate, phosphate, citrate, acetate, maleate, lactate, methanesulfonate, oxalate, fumarate, hydrobromide, p-toluenesulfonate, benzenesulfonate, or nitrate.

10. The water-soluble Nimodipine derivative or the pharmaceutically acceptable salt thereof according to claim 7, wherein
R₁ is selected from hydrogen, or Me; and
U together with V can form one of the following groups:

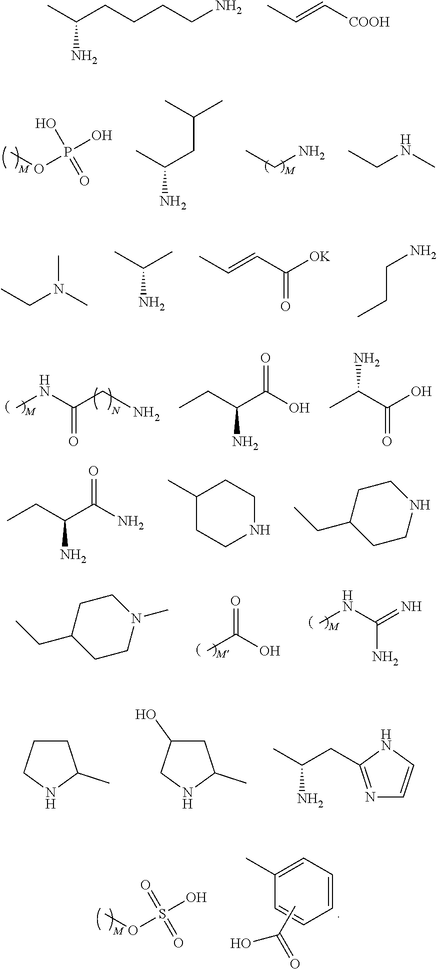

11. The water-soluble Nimodipine derivative or the pharmaceutically acceptable salt thereof according to claim 1, wherein the water-soluble Nimodipine derivative is selected from one of the following compounds: 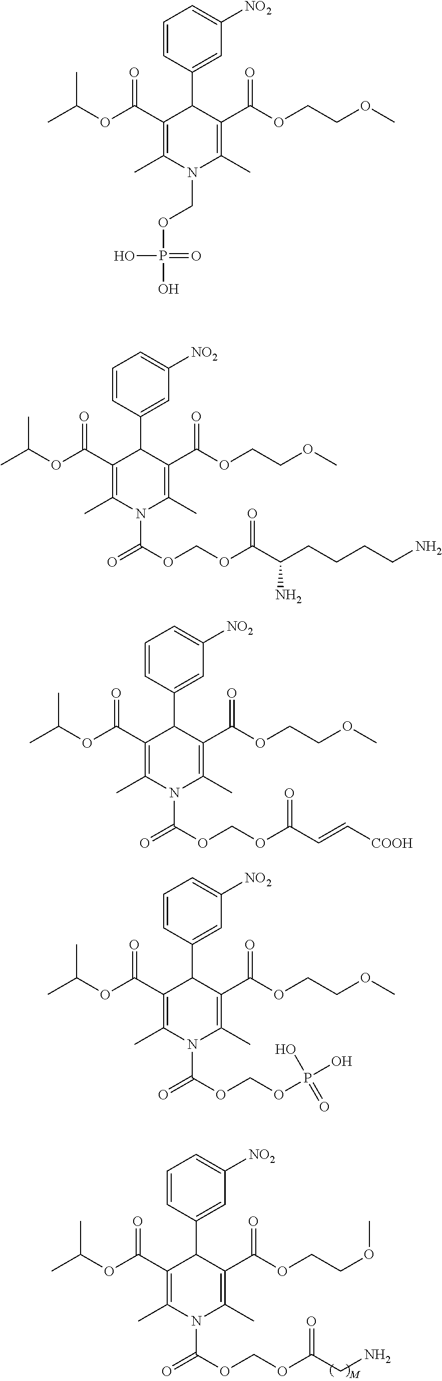 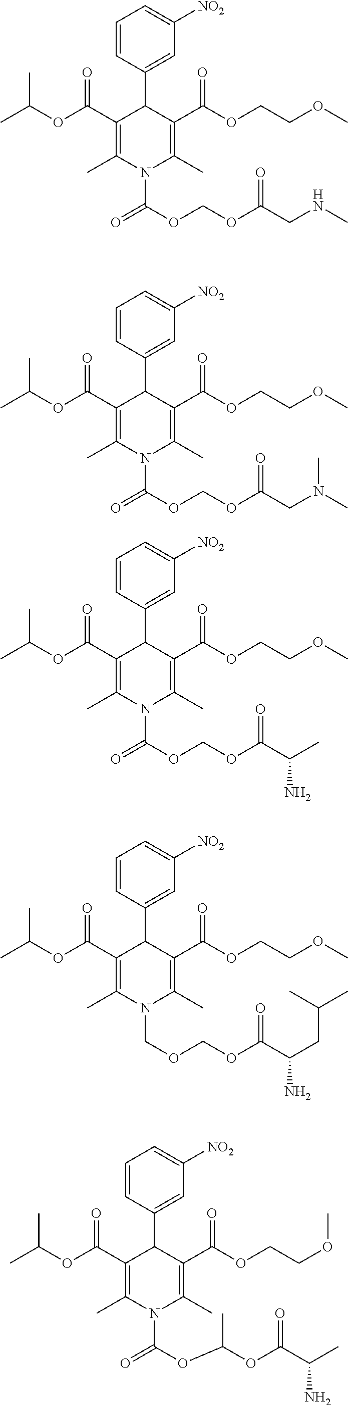 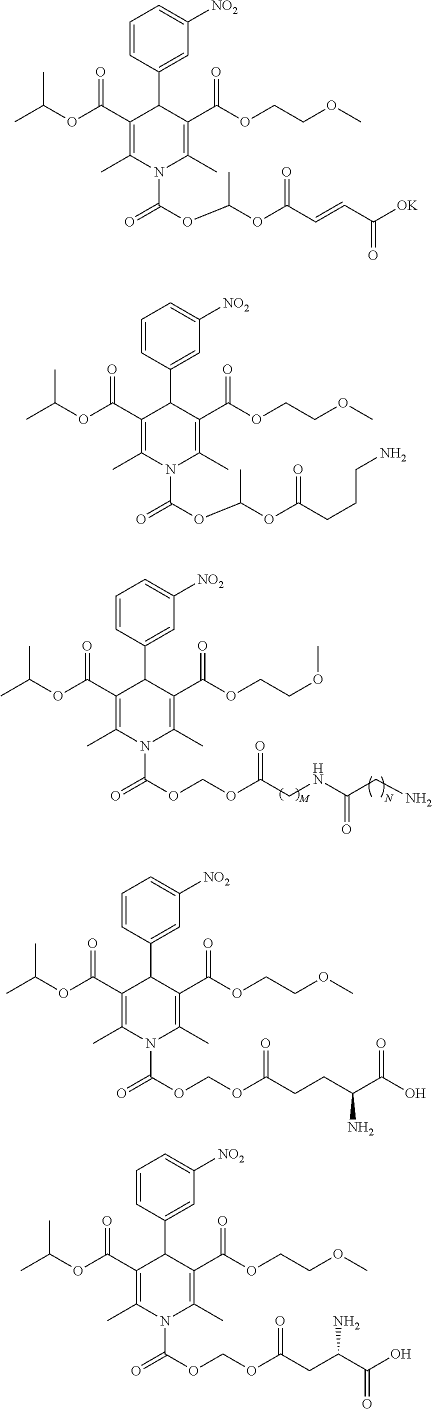 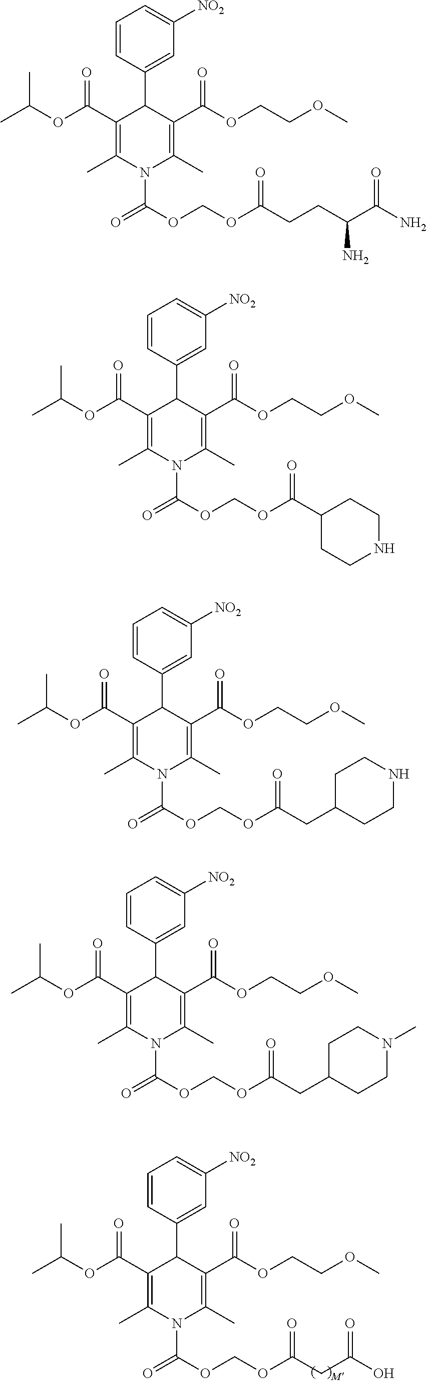 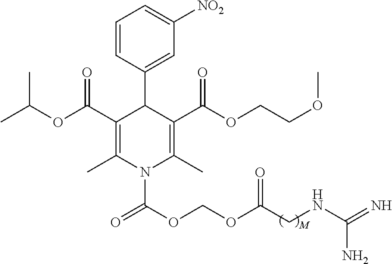
wherein M and N are independently selected from 1, 2, 3, 4, 5, or 6; and
M′ is selected from 0, 1, 2, 3, 4, 5, or 6.

12. A method of preparing the water-soluble Nimodipine derivative according to claim 7, wherein the method comprises:
reacting Nimodipine with halogenate chloroformate to form an amide; then reacting the amide with the corresponding carboxylic acid, amino acid or phosphoric acid derivative to form an ester, deprotecting, so as to yield the water-soluble Nimodipine derivative,
the reaction route is shown as below: 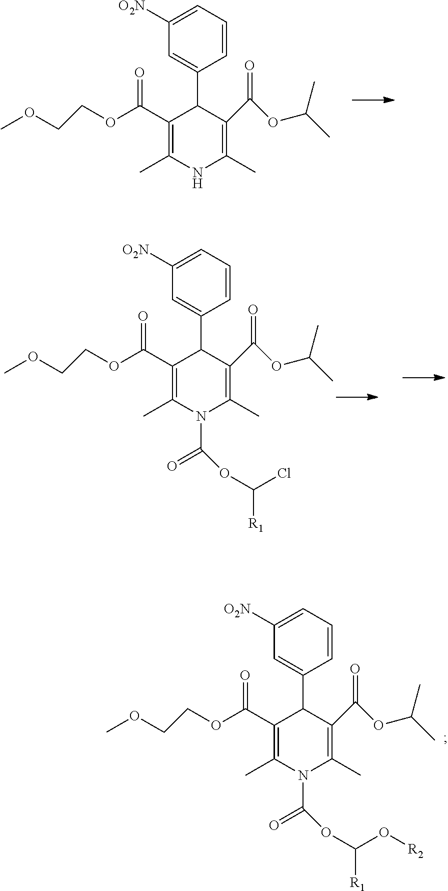
or
the method comprises:
reacting Nimodipine with di-tert-butyl chloromethyl phosphate to form a methylene phosphate, then deprotecting, so as to yield the water-soluble Nimodipine derivative,
the reaction route is shown as below.

13. A method for treating or preventing cardiovascular disease in a mammal in need thereof which comprises administering to the mammal an effective amount of the water-soluble Nimodipine derivative or the pharmaceutically acceptable salt thereof of claim 1.
